(19) United States
(12) Patent Application Publication
(10) Pub. No.: US 2022/0202768 A1
(43) Pub. Date: Jun. 30, 2022

(54) DIHYDROMYRICETIN NANOEMULSION FORMULATIONS AND METHODS FOR FORMING THEM

(71) Applicants: Cheers Health, Inc., Houston, TX (US); The Trustees of Princeton University, Princeton, NJ (US)

(72) Inventors: Robert K. PRUD'HOMME, Princeton, NJ (US); Brooks POWELL, Houston, TX (US); Chang TIAN, Princeton, NJ (US); Nicholas CAGGIANO, Princeton, NJ (US); Vikram PANSARE, Princeton, NJ (US)

(73) Assignees: Cheers Health, Inc., Houston, TX (US); The Trustees of Princeton University, Princeton, NJ (US)

(21) Appl. No.: 17/694,571

(22) Filed: Mar. 14, 2022

Related U.S. Application Data
(63) Continuation of application No. 16/723,127, filed on Dec. 20, 2019, now abandoned.
(60) Provisional application No. 62/786,058, filed on Dec. 28, 2018.

(51) Int. Cl. A61K 31/353; A61K 9/48; A61K 45/06; A61K 9/00; A61K 9/107

(52) U.S. Cl. CPC A61K 31/353; A61K 9/4825; A61K 9/4891; A61K 9/0053; A61K 9/107; A61K 45/06

(57) ABSTRACT

Nanoemulsion compositions, processes, and methods that include dihydromyricetin (DHM).

This application is a continuation of U.S. application Ser. No. 16/723,127, filed Dec. 20, 2019, which claims the benefit of U.S. Provisional Application No. 62/786,058, filed Dec. 28, 2018.

### FIELD OF THE INVENTION

The invention pertains to compositions, processes, and methods, including nanoemulsions, that include dihydromyricetin (DHM).

### BACKGROUND

Alcohol is a constituent of medicines, foods, and beverages that provides both beneficial and detrimental effects on human beings. Alcohol typically refers to ethyl alcohol (ethanol), which is the common form of consumable alcohol found in alcoholic beverages, e.g., such as beer, wine, and liquor. During consumption, alcohol is rapidly absorbed from the stomach and small intestine into the bloodstream, from which it can affect several organs including the brain, heart, pancreas, and liver. Alcohol can act as a depressant to the central nervous system (CNS). For example, alcohol interferes with the brain's communication pathways, which affects brain functionality that manifests in cognitive and behavioral changes, e.g., Such as a person's ability to think, focus, move, as well as his/her mood and behavior. Alcohol can cause inflammation and damage to the liver, e.g., where consistent heavy drinking can cause chronic liver problems. For example, heavy drinking can lead to steatosis (e.g., or fatty liver), infection (e.g., alcoholic hepatitis), fibrosis, and cirrhosis. More commonly, even a single instance of light to moderate to heavy alcohol consumption can result in what is commonly known as an ‘alcohol hangover’. A hangover refers to an array of physical symptoms that affect a person shortly after ingesting alcohol, e.g., within hours of consumption. The symptoms of a hangover include, for example, one or more of thirst, fatigue and/or weakness, headache and/or muscle aches, dizziness/faintness, loss of appetite, poor and/or decreased sleep, nausea and/or stomach pain (e.g., which can include vomiting), and elevated heart rate. A hangover is considered to be one of the most widely experienced negative consequences of consuming ethanol.[1]

### SUMMARY OF THE INVENTION

In an embodiment of the invention, a dihydromyricetin (DHM) pre-emulsion composition includes dihydromyricetin (DHM) and an emulsifier. The emulsifier can include a nonionic surfactant. The emulsifier can have a hydrophilic-lipophilic balance (HLB) greater than 7. The emulsifier can include a compound such as a polyethoxylated fatty acid or a polyethoxylated castor oil. The emulsifier can include a polymeric emulsifier, a polymer of ethylene oxide, a polyethylene glycol sorbitan fatty acid ester (polysorbate), polyoxyethylene sorbitan monolaurate (Tween 20), polyoxyethylene sorbitan monopalmitate (Tween 40), polyoxyethylene sorbitan monostearate (Tween 60), polyoxyethylene sorbitan tristearate (Tween 65), polyoxyethylene sorbitan mono-oleate (Tween 80), or combinations.

The emulsifier can include a polyethoxyethylene sorbitan monoester, a sugar surfactant, a sugar ester, a sugar fatty acid ester, sucrose monopalmitate, sucrose monolaurate, sucrose monostearate, sucrose distearate, sucrose monopalmitate, sucrose dipalmitate, sucrose monolaurate, saccharose monolaurate, a polyethylene glycol alkyl ether, a polyethylene glycol alkyl phenol, a polyglycerol ester, a polyethoxylated fatty acid diester, a polyethylene glycol fatty acid monoester, a polyethylene glycol fatty acid diester, a polyethylene glycol glycerol fatty acid ester, a fatty acid ester, a fatty alcohol ester, a carboxylic acid ester, a glycerol ester, a mixed ester of a fatty acid, a fatty alcohol, a carboxylic acid, and/or glycerol, and combinations, a citric acid ester of a monoglyceride, an alcohol oil transester, a polyvinyl alcohol, an alkyl cellulose, an alkyl guar, a hydroxyalkyl guar, a C₁-C₂ alkyl cellulose, a C₁-C₃ alkyl guar, a C₁-C₃ hydroxyalkyl guar, a polyvinylpyrrolidone, a polyvinylpyrrolidone copolymer, polyvinylcaprolactam, a polyvinylcaprolactam copolymer, a polyvinyl methyl ether, a polyvinyl methyl ether copolymer, an anionic surfactant, an anionic amphiphilic lipid, a polyacrylic acid, a polyacrylic acid copolymer, polyacrylic acid crosslinked with alkyl sucrose, polyacrylic acid crosslinked with allyl pentaerythritol, poly(acrylic acid-co-alkylacrylate), poly(acrylic acid-co-alkylacrylate) crosslinked with alkyl sucrose, poly(acrylic acid-co-alkylacrylate) crosslinked with allyl pentaerythritol, a polyacrylic acid salt (e.g., sodium polyacrylate), a polyacrylic acid copolymer salt, an alkyl ether citrate, an alkenyl succinate, an alkoxylated alkenyl succinate, an alkoxylated glucose alkenyl succinate, an alkoxylated methylglucose alkenyl succinate, a phosphoric acid fatty ester, alkaline salts of dicetyl and dimyristyl phosphate, alkaline salts of cholesterol sulfate, alkaline salts of cholesterol phosphate, lipoamino acids and their salts, mono- and disodium acylglutamates, disodium salt of N-stearoyl-L-glutamic acid, sodium salts of phosphatidic acid, phospholipids, an alkylsulfonic compound, an alkylsulfonic derivative, a cationic surfactant, a cationic amphiphilic lipid, a quaternary ammonium salt, a fatty amine, a primary fatty amine, a secondary fatty amine, a tertiary fatty amine, a fatty amine salt, a primary fatty amine salt, a secondary fatty amine salt, a tertiary fatty amine salt, or combinations.

The DHM pre-emulsion composition can include a co-surfactant. For example, the co-surfactant can be 2(2-ethoxyethoxy)ethanol, a sorbitan fatty acid ester, sorbitan monolaurate, sorbitan monopalmitate, sorbitan tristearate, sorbitan monostearate, sorbitan monooleate, sorbitan trioleate, a phospholipid, egg lecithin, soy lecithin, epikuron, topcithin, leciprime, lecisoy, emulfluid, emulpur, metarin, emultop, lecigran, lecimulthin, hydroxylated lecithin, lysophosphatidylcholine, cardiolipin, sphingomyelin, phosphatidylcholine, phosphatidyl ethanolamine, phosphatidic acid, phosphatidyl glycerol, phosphatidyl serine, an ionic surfactant, sodium stearoyl lactylate, calcium stearoyl lactylate, or combinations.

The DHM pre-emulsion composition can further include an oil. The oil can have a molecular weight of less than 400 Da. In the DHM pre-emulsion composition, the DHM can be dispersed or dissolved in the oil.

The DHM pre-emulsion composition can include a permeabilizer. The permeabilizer can include capric acid, a caprate salt, and/or sodium caprate. The permeabilizer can include a fatty acid, a saturated fatty acid, and/or a fatty acid complexed with a cation such as a metal cation, a metal divalent cation, a magnesium divalent cation, a calcium divalent cation, a zinc divalent cation, an iron divalent cation, a metal trivalent cation, an iron trivalent cation, a fatty acid salt, a fatty acid metallic soap, or combinations.

The DHM pre-emulsion composition can further an antioxidant. The DHM pre-emulsion composition can include an electrolyte and/or a sugar. The DHM pre-emulsion composition can include a coactive, for example, glutathione, L-cysteine, N-acetyl cysteine (NAC), Prickly Pear extract, Milk Thistle, ginger root, vitamin B, vitamin C, vitamin E, and combinations.

In an embodiment of the invention, the DHM pre-emulsion can disperse to emulsion droplets of a mean diameter of at most 1000 nanometers when contacted with an excess aqueous phase.

In an embodiment of the invention, a dosage form includes the dihydromyricetin (DHM) pre-emulsion composition of the invention and a capsule, and the DHM pre-emulsion composition is encapsulated in the capsule. The capsule can be a soft gel capsule. The capsule can include a material such as animal-derived material, gelatin, collagen, plant-derived material, synthetically-produced material, a polysaccharide, a sulfated polysaccharide, a carrageenan, cellulose, a cellulose derivative, starch, a starch derivative, pullulan, polyvinyl alcohol (PVA), polyvinyl alcohol (PVA) copolymer, polyethylene glycol (PEG), hydroxypropyl methylcellulose

(HPMC), hydroxypropyl methyl cellulose acetate succinate (HPMCAS), a material of algal origin, or combinations. The capsule is not solubilized or dissolved by an aqueous solution having a pH of at most 3.5, and the capsule is solubilized or dissolved by an aqueous solution having a pH of at least 5.5. The capsule can include an exterior surface and the exterior surface can be coated with an enteric coating. The enteric coating can be a polymeric coating, a methacrylate copolymer coating, a hydroxypropyl methyl cellulose acetate succinate (HPMCAS) coating, or combinations.

In an embodiment of the invention, a dihydromyricetin (DHM) pre-emulsion composition includes dihydromyricetin (DHM); polyoxyethylene sorbitan monolaurate; capric acid, sodium caprate, or a caprate salt; and soybean oil. The dihydromyricetin (DHM) can be at a concentration of from 3 to 30 mg/mL, the polyoxyethylene sorbitan monolaurate can be at a concentration of from 0.3 to 3 mg/mL, and the capric acid, sodium caprate, or caprate salt can be at a concentration of from 0.02 mg/mL to 0.2 mg/mL in the soybean oil.

In an embodiment of the invention, a dihydromyricetin (DHM) pre-emulsion composition includes dihydromyricetin (DHM), polyethoxylated castor oil, 2,3-dihydroxypropyl octanoate, and 2-(2-ethoxyethoxy)ethanol. The dihydromyricetin (DHM) pre-emulsion composition can further include capric acid, sodium caprate, and/or a caprate salt. The dihydromyricetin (DHM) pre-emulsion composition can further include water. The dihydromyricetin (DHM) pre-emulsion composition of claim 27 can include from 10 wt % to 24 wt % dihydromyricetin (DHM), from 9 wt % to 21 wt % 2,3-dihydroxypropyl octanoate, from 9 wt % to 21 wt % 2-(2-ethoxyethoxy)ethanol, and from 0 wt % to 34 wt % capric acid, sodium caprate, and/or a caprate salt, and the balance can include polyethoxylated castor oil. The dihydromyricetin (DHM) pre-emulsion composition of can include from 2 wt % to 20 wt % dihydromyricetin (DHM), from 7 wt % to 20 wt % 2,3-dihydroxypropyl octanoate, from 3 wt % to 20 wt % 2-(2-ethoxyethoxy)ethanol, and from 14 wt % to 40 wt % polyethoxylated castor oil, and the balance the balance can include water.

A method for forming the dosage form of the invention includes mixing the dihydromyricetin (DHM), the emulsifier, and an oil to dissolve or disperse the DHM and the emulsifier in the oil to form the DHM pre-emulsion composition; loading the DHM pre-emulsion composition into the capsule; and sealing the capsule.

A method for administering dihydromyricetin (DHM) to a patient includes orally administering the dosage form of the invention to the patient, allowing the capsule to enter the patient's stomach, where the capsule is not dissolved and is not solubilized by gastric juices in the stomach, allowing the capsule to pass from the stomach to the patient's intestine, where the capsule is dissolved or solubilized by intestinal fluid in the intestine, allowing the partially or fully dissolved or solubilized capsule to release the DHM pre-emulsion composition into the intestinal fluid, allowing the released DHM pre-emulsion composition to form a metastable nanoemulsion comprising oil-in-water droplets in the intestinal fluid, and allowing the DHM to diffuse from the oil-in-water droplets into a wall of the intestine and into the patient's bloodstream, so that the DHM is administered to the patient.

A method for reducing hangover symptoms includes administering the dihydromyricetin (DHM) pre-emulsion composition of the invention to a patient suffering from hangover symptoms, so that the patient's hangover symptoms are reduced.

The dihydromyricetin (DHM) formulation of the invention can be used in preventing an alcohol use disorder, preventing alcoholism, treating an alcohol use disorder, treating alcoholism, or treating an alcohol overdose. For example, the dihydromyricetin (DHM) formulation of the invention can be administered to a patient at risk for developing an alcohol use disorder or alcoholism, so that the development of an alcohol use disorder or alcoholism in the patient is prevented or delayed or the risk of development of an alcohol use disorder or alcoholism in the patient is diminished. For example, the dihydromyricetin (DHM) formulation of the invention can be administered to a patient suffering from an alcohol use disorder or alcoholism, so that the alcohol use disorder or alcoholism of the patient is cured or ameliorated. For example, the dihydromyricetin (DHM) formulation of the invention can be administered to a patient suffering from an alcohol overdose, so that the effects of the alcohol overdose are prevented, reversed, or ameliorated. The dihydromyricetin (DHM) formulation of the invention can be used in increasing antioxidant capacity, neuroprotection, preventing Alzheimer's disease, treating Alzheimer's disease, inhibiting inflammation, protecting the kidney, protecting the liver, preventing or treating cancer, ameliorating a metabolic disorder, preventing diabetes, treating diabetes, or treating a bacterial infection. For example, the dihydromyricetin (DHM) formulation of the invention can be administered to a patient in need of increased antioxidant capacity, neuroprotection, kidney protection, or liver protection, so that the patient's antioxidant capacity is increased, the patient experiences neuroprotection, and or the patient's kidney or liver is protected. For example, the dihydromyricetin (DHM) formulation of the invention can be administered to a patient at risk of developing Alzheimer's disease, inflammation, cancer, or diabetes, so that the development of Alzheimer's disease, inflammation, cancer, or diabetes is prevented or delayed or the risk of development of Alzheimer's disease, inflammation, cancer, or diabetes in the patient is diminished. For example, the dihydromyricetin (DHM) formulation of the invention can be administered to a patient suffering from Alzheimer's disease, cancer, a metabolic disorder, diabetes, or a bacterial infection, so that the patient's Alzheimer's disease, cancer, metabolic disorder, diabetes, or bacterial infection is cured, ameliorated, or its progression is delayed.

### BRIEF DESCRIPTION OF THE FIGURES

FIG. 1 is a graph that presents the particle size distribution in nanoemulsions of the formulation NE-1A as Intensity versus Diameter for successively greater dilutions with water of 1:19, 1:39, 1:79, and 1:159. For example, dilution of 80 times (dilution with NE-1A:water of 1:79) yields particles having a mean equilibrium diameter of about 200 nm.

FIG. 2 is a graph that presents the particle size distribution in nanoemulsions of the formulation NE-2A as Intensity versus Diameter for successively greater dilutions with water of 1:19, 1:39, 1:79, and 1:159. For example, dilution of 20 times (dilution with NE-2A:water of 1:19) yields particles having a mean equilibrium diameter of about 300 nm.

FIG. 3 is a graph that presents the particle size distribution in a nanoemulsion of the formulation NE-3 as Intensity versus Diameter for a dilution with water of 1:449. That is, dilution of 500 times (dilution with NE-3:water of 1:499) yields particles having a mean equilibrium diameter of about 70 nm. FIG. 4 is a graph that presents the particle size distribution in a nanoemulsion of the formulation NE-4 as Intensity versus Diameter for a dilution with water of 1:449. That is, dilution of 500 times (dilution with NE-4:water of 1:499) yields particles having a mean equilibrium diameter of about 80 nm.

FIG. 5 is a graph that illustrates how nanodroplets formed from the NE-5 formulation release DHM into FaSSIF. The graph shows that about 70% of the DHM in the nanodroplets is gradually released over 6 hours from the nanodroplets into FaSSIF.

FIG. 6 is a graph that illustrates how nanodroplets formed from the NE-5 formulation release DHM into FeSSIF. The graph shows that about 60% of the DHM in the nanodroplets is gradually released over 6 hours from the nanodroplets into FeSSIF.

FIG. 7 is a graph that illustrates in vivo uptake of DHM into the bloodstream from a no-water formulation without capric acid as Concentration of DHM in blood plasma versus Time after the administration of the formulation. For each sample, blood was drawn by a catheter from the tail vein of a rat. Plasma is prepared from blood and the amount of DHM is determined by LC/MS (liquid chromatography/mass spectrometry).

FIG. 8 is a graph that demonstrates in vivo uptake of DHM into the bloodstream from a no-water formulation with capric acid as Concentration of DHM in blood plasma versus Time after the administration of the formulation. For each sample, blood is drawn by a catheter from the tail vein of a rat. Plasma is prepared from blood and the amount of DHM is determined by LC/MS.

### DETAILED DESCRIPTION OF THE INVENTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent parts can be employed and other methods developed without parting from the spirit and scope of the invention. All references cited herein are hereby incorporated by reference in their entirety as if each had been individually incorporated.

An aspect of the present invention includes a method to improve the bioavailability of the molecule dihydromyricetin (DHM) through the process known as nano-emulsification. This method can include processing by nano-emulsification of a combination of materials including DHM, surfactants and/or emulsifiers, and oils, and optionally including additional beneficial molecules (e.g., co-actives) and/or permeabilizers. In an embodiment, the DHM, surfactants and/or emulsifiers, and oils, and optionally the additional beneficial molecules (e.g., co-actives) and/or permeabilizers, are dissolved or dispersed together and filled into a soft gel capsule for administration. Upon ingestion, the gel capsule can dissolve or solubilize, releasing the oil contents, with the materials dissolved or dispersed therein, and generating the nano-emulsion spontaneously within the gastrointestinal tract, gut, stomach, intestines, small intestine, and/or large intestine. The final form of the product can include such an oil or a similar gel (the gel including such an oil) to be placed within a gel capsule for administration. The final form of the product can include a gel capsule containing an oil or a similar gel (the gel including such an oil) for administration. These final forms of product can provide improvements in bioavailability and pharmacokinetic parameters. In some embodiments, a formulation according to the invention may be especially well suited to oral administration routes. For example, the capsule or gel capsule may be of or include material of algal origin; i.e., the material of which the wall of the capsule is formed may be of or include material of algal origin or derived from an algal material.

Weder and Mutsch (U.S. Pat. No. 5,152,923A, 1992) discuss nanoemulsions containing sub-200 nm sized oil droplets comprising a triglyceride or fatty acid ester in an aqueous phase produced by a high-pressure homogenizer.[15] Simonnet, et al. (U.S. Pat. No. 6,689,371B1, 2004) discusses nanoemulsions including an oily phase dispersed in an aqueous phase with globules less than 100 nm in size, a surfactant that is solid below 45° C. selected from the group consisting of esters of a fatty acid and of a sugar, and ethers of a fatty alcohol and of a sugar, an oil with a molecular weight greater than 400 Daltons, and a 2:10 mass ratio of oil to surfactant; it discusses vigorous mixing and high-pressure homogenization to produce the nanometer-sized oil globules.[16] Simonnet, et al. (U.S. Pat. No. 6,335,022B1, 2002) discusses nanoemulsions including oil globules less than 100 nm (nanometers) in size on average, a surfactant that is solid below 45° C. from a group of sorbitan fatty esters, an oil with a molecular weight greater than 400

Daltons, and at least one ionic amphiphilic lipid chosen from a specified group of compounds including dicetyl and dimyristyl phosphates; it discusses the use of vigorous mixing and high pressure-homogenization to produce the nanometer-sized oil globules.[17] Simonnet, et al. (U.S. Pat. No. 6,274,150B1, 2001) discusses nanoemulsions including oil globules less than 100 nm in size on average, an anionic surfactant selected from a group including phosphoric acid fatty esters and oxyethylenated derivatives, and an oil with molecular weight greater than 400 Daltons; it discusses the use of vigorous mixing and high-pressure homogenization to produce the nanometer sized oil globules.[18] Simonnet, et al. (U.S. Pat. No. 6,375,960B1, 2002) discusses nanoemulsions including oil globules less than 100 nm in size on average, an ethoxylated fatty ether or ester surfactant that is solid below 45° C., and an oil with a molecular weight greater than 400 Daltons; it discusses the use of vigorous mixing and high-pressure homogenization to produce the nanometer-sized oil globules.[19] L'Alloret (U.S. Pat. No. 6,998,426B2, 2006) discusses the preparation of nanoemulsions including nonionic or anionic amphiphilic lipids, at least one water soluble nonionic polymer, and an oil phase, in order to provide rheological modifications; the nonionic polymer increases viscosity through gelation of the aqueous phase; it discusses the use of vigorous mixing and high-pressure homogenization to produce the nanometer-sized oil globules.[20] Quemin (U.S. Pat. No. 6,902,737B2, 2005) discusses nanoemulsions including oily globules less than 100 nm in size on average in a ternary surfactant system; two of the surfactants are nonionic and include at least one ethoxylated fatty ester and at least one fatty acid ester of sorbitan; the other surfactant is an ionic surfactant chosen from alkali metal salts of cetyl phosphate and alkali metal salts of palmitoyl sarcosinate.[21] Simonnet, et al. (U.S. Pat. No. 6,413,527, 2002) discusses nanoemulsions including oil globules less than 100 nm in size on average, an anionic surfactant selected from the group including alkyl ether citrates, and an oil with a molecular weight greater than 400 Daltons; it discusses the use of vigorous mixing and high-pressure homogenization to produce the nanometer-sized oil globules.[22] Wooster, et al. (International Applic. Publication WO2009067734A1, 2009) discusses the preparation of oil-in-water nanoemulsions by mechanical means with up to 40 wt % oil and a triglyceride surfactant with a fatty acid chain length of 12 carbon atoms or greater, and possibly a co-surfactant; the preparation process involves high-energy sonication or high-shear homogenization.[23] Nicolosi, et al. (US Published Pat. Applic. 20110206739A1, 2011) discusses the formation of nanoemulsions including oils, water, and one or more surfactants capable of causing a temperature-dependent phase inversion to form oil globules less than 100 nm in size without high-energy homogenization.[24] Ochomogo (US Published Pat. Applic. 20130064954A1, 2013) and (US Published Pat. Applic. 20110229516A1, 2011) discuss the preparation of nanoemulsions by low-energy phase inversion methods; formulations include an active ingredient, one or more surfactants which may be ionic or nonionic, and water, for oral or nasal delivery.[25, 26] Brito, et al. (International Applic. Publication WO2015140138A1, 2015) discusses oil-in-water emulsions prepared by phase-inversion driven by composition changes in the oil/water ratio to form oil droplets of less than 250 nm or less than 40 nm size; the hydrophilic-lipophilic balance (HLB) of the surfactant(s) is varied to create adjuvants.[27] Zhou, et al. (U.S. Pat. No. 7,977,024B2, 2011) discusses a process for making toner particles for printing applications including an emulsion formulation; the phase-inversion emulsification process is temperature-driven, and provides a toner composition including a toner resin, a wax, and a charge control agent; the toner resin includes an epoxy resin and a sulfonated polyester resin; process temperatures range from 50° C. to about 120° C. to form a molten toner composition which is then emulsified.[28]

Formulations according to the invention can include, but are not limited to, a combination of materials including the active ingredient, such as the flavonoid DHM, additional beneficial active molecules (co-actives), permeability enhancers, emulsifiers (surfactants), oils, and gel capsules. The gel capsules may further be coated with an enteric coating to improve stability. For example, the capsule or gel capsule may be of or include material of algal origin; i.e., the material of which the wall of the capsule is formed may be of or include material of algal origin or derived from an algal material.

### DHM

Dihydromyricetin (DHM), a flavonoid compound isolated from the Hovenia plant can “sober-up” rats inebriated with alcohol[2], prevent predisposed rats from becoming alcoholics[2], return alcoholic rats to baseline levels of alcohol consumption[2], reduce hangover symptoms[2], and prevent fetal alcohol spectrum disorders in the offspring of rats exposed to significant amounts alcohol during pregnancy.[2] DHM can be dissolved in a solvent, such as dimethylsulfoxide (DMSO). DHM can be complexed with a metal, such as a divalent alkali earth metal, divalent magnesium (Mg(II), Mg⁺²), a divalent transition metal, divalent iron (Fe(II), Fe²), divalent copper (Cu(II), Cu²), a trivalent transition metal, or trivalent iron (Fe(III), Fe³).

DHM has unique physicochemical properties including low solubility and high hydroxyl functional group content, rendering the processing of DHM and other flavonoids to produce formulations for therapeutic administration and to improve their dissolution kinetics and bioavailability difficult.

DHM demonstrates the pharmacological properties expected to underlie successful medical treatment of alcohol use disorders (AUDs) [29-31]. Given limited available pharmacotherapies for AUDs and these being limited by low patient compliance, because of the adverse effects they cause, therapies for the treatment of AUDs should be advanced, e.g., through DHM therapeutic strategies. [32]

In addition to DHMs potential for the treatment of AUDs, which, without being bound by theory, may be achieved through DHM's inhibiting the effect of alcohol on GABAA receptors (GABAARs) in the brain, DHM and the Hovenia plant it is isolated from have shown efficacy in mitigating liver injuries [33-35], decreasing alcohol and acetaldehyde concentrations in the blood via enhancing ADH and ALDH activity [36,37], and eliminating alcohol-induced excessive free radicals. [38] DHM has been observed to have oxidative stress-mediating activity, i.e., increase antioxidant capacity for scavenging reactive oxygen species, which may result in neuroprotective, nephroprotective (kidney protecting), and hepatoprotective (liver protecting) effects, which may ameliorate, for example, the effects of hypobaric hypoxia, side effects of the chemotherapeutic agent cisplatin, and detrimental effects of ethanol. DHM may have a neuroprotective role in Alzheimer's and Parkinson's diseases. DHM can also inhibit inflammation. DHM can also have anticancer activity and regulate cell proliferation and apoptosis. DHM can mediate metabolism, and may be useful in ameliorating certain metabolic disorders, such as diabetes, weight gain, hyperlipidemia, and atherosclerosis. DHM exhibits antibacterial activity (Li, H. et al., “The Versatile Effects of Dihydromyricetin in Health”, EvidenceBased Complementary & Alternative Medicine 2017, Art. ID 1 053617).

A DHM formulation designed to reduce alcohol's negative effects when taken after alcohol consumption is covered under U.S. Pat. No. 9,603,830 B2 (granted on Mar. 28, 2017) and is sold in the US under the brand name Thrive+®.

Despite promising results in rats, one challenge in translating DHM's efficacy to humans in a commercially viable way is DHM's oral bioavailability of less than 5% [39]. DHM is a BCS class IV drug limited by having the properties of both low solubility and permeability. In the context of successfully commercialized drugs, DHM requires large doses for efficacy.

This invention addresses the problem of poor bioavailability and stability of DHM through the formation of nanoemulsions. The pre-emulsion composition, formed of DHM and an emulsifier (surfactant) dispersed or dissolved in oil and, for example, encapsulated within a gel capsule, is thermodynamically stable. By dispersing or dissolving DHM and emulsifiers within an oil phase, upon dissolution or solubilization of the gel capsule that the mixture is placed in, the oil will rapidly break up into droplets that can be less than 200 nm in size and are stabilized by the emulsifiers. For example, the emulsion droplets formed can be at most 10,000 nm, 3000 nm, 1000 nm, 400 nm, 200 nm, or 100 nm in diameter. By preparing and applying such a controlled-release DHM nanoemulsion, DHM may exhibit enhanced dissolution and release kinetics, and higher concentrations in the body. The encapsulating gel capsule further provides improved stability when the dosage form is exposed to low pH gastric juices and enzymes, which can cause degradation and quenching of DHM activity, than when DHM is administered in a pure form. Furthermore, the DHM nanoemulsion formulation may possess an improved ability to penetrate intestinal barriers, to allow DHM to reach the bloodstream more effectively and efficiently. For example, the capsule or gel capsule may be of or include material of algal origin; i.e., the material of which the wall of the capsule is formed may be of or include material of algal origin or derived from an algal material.

In addition to DHM, a formulation according to the invention can include additional beneficial molecules (co-actives), such as glutathione, L-cysteine, N-acetyl cysteine (NAC), Prickly Pear extract, Milk Thistle, ginger root, vitamin B, vitamin C, vitamine E, electrolytes, and/or sugars.

### Emulsifiers

A nanoemulsion includes oil, water, and one or more emulsifiers (surfactants). The addition of an emulsifier is required to create the small (nano-sized) droplets in a nanoemulsion, because the emulsifier decreases the interfacial tension, i.e., the surface energy per unit area, between the oil and water phases of the emulsion. The emulsifier also stabilizes nanoemulsions through repulsive electrostatic interactions and steric hindrance. Unless otherwise indicated, the terms emulsifier and surfactant are used interchangeably herein. The emulsifier used can be a small-molecule surfactant, e.g., an artificially synthesized small-molecule surfactant, but proteins, lipids, fatty acids, and polymers can also be used in the preparation of nanoemulsions.[14] Excipients are materials which aid in the formulation, stability, and/or release characteristics of the active molecule DHM; an emulsifier is a class of excipient. For example, an emulsifier can have a hydrophilic-lipophilic balance (HLB) of from 6 to 18, from 7 to 18, from 7 to 16, or from 8 to 16.

Examples of emulsifiers follow:

Polymeric emulsifiers: homopolymers and copolymers of ethylene oxide; polyvinyl alcohols; homopolymers and copolymers of vinylpyrrolidone; homopolymers and copolymers of vinylcaprolactam; homopolymers and copolymers of polyvinyl methyl ether; neutral acrylic homopolymers and copolymers; C₁-C₂ alkyl celluloses and their derivatives; C₁-C₃ alkyl guar; C₁-C₃ hydroxyalkyl guar; and combinations and derivatives thereof.

Nonionic surfactants and polymers: The hydrophilic non-ionic surfactant can have a hydrophilic-lipophilic balance (HLB) greater than 7 and can be a food grade or pharmaceutical grade hydrophilic surfactant such as polysorbates (polyethylene glycol sorbitan fatty acid esters), polyethylene glycol alkyl ethers, sugar esters, polyethoxylated fatty acids, polyoxyethylene-polyoxypropylene block co-polymers (Pluronics), polyethylene glycol alkyl phenol surfactants, citric acid esters of monoglycerides, polyglycerol esters, polyethoxylated fatty acid diesters,

PEG- fatty acid mono and diesters, polyethylene glycol glycerol fatty acid esters and alcohol oil transesters or mixtures thereof. Suitable non-ionic surfactants include: polysorbates for example polyethoxyethylene sorbitan monoesters including polyoxyethylene sorbitan monolaurate (Tween 20), polyoxyethylene sorbitan monopalmitate (Tween 40) , polyoxyethylene sorbitan monostearate (Tween 60) , polyoxyethylene sorbitan tristearate (Tween 65) and polyoxyethylene sorbitan mono- oleate (Tween 80) ; sugar surfactants, for example, sucrose monopalmitate, sucrose monolaurate, sucrose distearate 3 Crodesta F-10, sucrose distearate, monostearate Crodesta F-110, sucrose dipalmitate, sucrose monostearate Crodesta F-160, sucrose monopalmitate, sucrose monolaurate and saccharose monolaurate; polyoxyethylene-polyoxypropylene block copolymers which are available under various trade names including Synperonic PE series (ICI), Pluronic .RTM. series (BASF) , Emkalyx, Lutrol (BASF), Supronic, Monolan, Pluracare and Plurodac, polyethoxylated castor oil (Kolliphor EL, Cremophor EL).

The polyoxyethylene-polyoxypropylene block copolymers are also known as “polyoxamers” and have the general formula: HO(C₂H₄O)_{A}(C₃H₆O)_{B}(C₂H₄O)_{A}H in which A and B denote the number of polyoxyethylene and polyoxypropylene units, respectively. Polyoxamers when A is 1-100 and B is 1-100 and combinations thereof are suitable for use in the nanoemulsions of the present invention.

The anionic amphiphilic lipids which can be used in the nanoemulsions of the invention can be chosen from, for example, 1) mixed esters of fatty acid or of fatty alcohol, of carboxylic acid and of glycerol, 2) alkyl ether citrates, 3) alkenyl succinates chosen from alkoxylated alkenyl succinates, alkoxylated glucose alkenyl succinates and alkoxylated methylglucose alkenyl succinates, and 4) phosphoric acid fatty esters. Polymers marketed under the trade name Carbopol (Lubrizol) may be included, for example, polyacrylic acid crosslinked with alkyl sucrose, polyacrylic acid crosslinked with allyl pentaerythritol, poly(acrylic acid-co-alkylacrylate) crosslinked with allyl pentaerythritol. Other polymers include polyacrylic acid, a polyacrylic acid copolymer, poly(acrylic acid-co-alkylacrylate), poly(acrylic acid-co-alkylacrylate) crosslinked with alkyl sucrose, a polyacrylic acid salt (e.g., sodium polyacrylate), and a polyacrylic acid copolymer salt. Additional anionic emulsifiers include alkaline salts of dicetyl and dimyristyl phosphate; alkaline salts of cholesterol sulfate; alkaline salts of cholesterol phosphate; lipoamino acids and their salts, such as mono- and disodium acylglutamates, for instance the disodium salt of N-stearoyl-L-glutamic acid sold under the name Acylglutamate HS21 by Ajinomoto; sodium salts of phosphatidic acid; phospholipids; and alkylsulfonic derivatives.

The cationic amphiphilic lipids which may be used in the nanoemulsions of the invention can be, for example, chosen from the group formed by quaternary ammonium salts, fatty amines and salts and other derivatives thereof.

The nanoemulsion may also contain a co-surfactant which can be a surfactant that acts synergistically with a hydrophilic non-ionic surfactant to alter the interfacial curvature. This lowers interfacial tension, permitting easier emulsion formation. For example, the co-surfactant can be food grade or pharmaceutical grade. Suitable food grade co-surfactants include, but are not limited to: transcutol (2-(2-ethoxyethoxy)ethanol), sorbitan fatty acid esters such as sorbitan monolaurate (Span 20), sorbitan monopalmitate (Span 40), sorbitan tristearate (Span 65), sorbitan monostearate (Span 60) , sorbitan monooleate (Span-80) and sorbitan trioleate (Span-85); phospholipids such as egg/soy lecithin for example epikuron, topcithin, leciprime, lecisoy, emulfluid, emulpur, metarin, emultop, lecigran, lecimulthin, ovothin lyso egg/soy lecithin, hydroxylated lecithin lysophosphatidylcholine, cardiolipin, sphingomyelin , phosphatidylcholine, phosphatidyl ethanolamine, phosphatidic acid, phosphatidyl glycerol, phosphatidyl serine and mixtures of phospholipids with other surfactants; and ionic surfactants such as sodium stearoyl lactylate and calcium stearoyl lactylate.

In an embodiment of the invention, the following components can be used as emulsifiers: surfactants, such as ethoxylated sorbitan esters, e.g., Tween 60 (ethoxylated-20 sorbitan monostearate, polyoxyethylene (20) sorbitan monostearate) and Tween 80 (ethoxylated-20 sorbitan monooleate, polyoxyethylene (20) sorbitan monooleate) and sucrose ester surfactants (sucrose alkanoates) which are mono-, di-, tri and polyesters of sucrose (sucrose mono-, di-, and polyalkanoates), fatty acids, oils of limonene, and alcohols including ethanol, n-butanol, and 1,2-propanediol.

### Oils

The oil can be a hydrophobic and/or lipophilic liquid that may be immiscible with the water phase and provides a medium for dissolution of the active molecules (e.g., DHM), emulsifiers, permeabilizers, and other excipients and materials.

For example, oils with molecular weight greater than or equal to 400 Daltons (Da) can be used to provide improved stability. Such oils with a molecular weight of greater than or equal to 400 Da can be chosen from oils of animal or vegetable origin, mineral oils, white oils, synthetic oils and silicone oils, and their mixtures. Examples of such oils include isocetyl palmitate, isocetyl stearate, avocado oil or jojoba oil.

For example, the oily phase can optionally comprise other oils and in particular oils having a molecular weight of less than 400 Da. Such oils can be selected from oils of animal or vegetable origin, mineral oils, white oils, synthetic oils and silicone oils. Examples are oils with a molecular weight of less than 400, such as Capmul MCM (2,3-dihydroxypropyl octanoate) isododecane, isohexadecane, volatile silicone oils, isopropyl myristate, isopropyl palmitate, and C₁₁-C₁₃ isoparaffin.

The oil can include straight chain or branched saturated alkanes, alkenes, or alkynes.

The oily phase can include fatty substances other than the oils indicated above, such as fatty alcohols, for example, stearyl, cetyl, and behenyl alcohols, fatty acids, for example, stearic, palmitic, and behenic acids, oils of a fluorinated type, waxes, gums, and mixtures thereof. Long chain triglycerides may also be included, such as those of animal origin such as fish oil, cod liver oil, blubber, lard, tallow, schmaltz, and butter fat; vegetable origin such as canola oil, castor oil, cocoa butter, coconut oil, coffee seed oil, corn oil, cotton seed oil, evening primrose oil, grapeseed oil, flax seed oil, menhaden oil, mustard seed oil, olive oil, palm oil, palm kernel oil, peanut oil, poppy seed oil, rapeseed oil, rice bran oil, safflower oil, sesame oil, soybean oil, sunflower oil, palm kernel oil, hazelnut oil, sesame oil and wheat germ oil; oil of algal origin; vegetable oil; and combinations of these. Synthetic triglycerides, fractionated triglycerides, modified triglycerides, hydrogenated triglycerides or partially hydrogenated and mixtures of triglycerides are also included.

The nanoemulsion formulation may contain one or more additional oils such as short chain triglycerides, for example, triacetin, tributyrin, tricapylrin and miglyol; mineral oils, for example, alkane oils such as decane, tetradecane, hexadecane and octadecane; and flavour oils, for example, limonene, mandarin oil orange oil, lemon oil, lime oil or other citrus oils, peppermint oil, peach oil, vanilla flavour oil and vanillin; and aromatic oils, for example, peppermint, tea tree oil, eucalyptus oil, mentha arvensis, cedarwood oil, spearmint, orange oil, lemon oil, and clove oil. The ratio of triglyceride to additional oil can be from 1:0 to 1:1.

### Permeabilizers

A permeability-enhancer or permeabilizer is an agent (e.g., a chemical compound) that enhances the permeation (increases the rate of transport) of a drug compound through (across) the epithelial cell layer in the gastrointestinal (GI) tract and thereby enhances the amount of drug entering the bloodstream. Permeability-enhancers have been reviewed by Aungst and Whitehead[18-21]. The list of agents presented by Aungst in Table I and Whitehead in Table I are incorporated into this application in their entirety.

Examples of permeability-enhancers are fatty acids, a saturated fatty acid, capric (decanoic) acid, a caprate salt, sodium caprate, caprylic (octanoic) acid, a caprylate salt, sodium caprylate, a fatty acid complexed with a cation, such as a metal cation, magnesium (Mg), calcium (Ca), zinc divalent cation (Zn(II), Zn⁺²), iron divalent cation (Fe(II), Fe⁺²), iron trivalent cation (Fe(III), Fe⁺³), or combinations. For example, capric acid and its salts are permeabilizers that are currently clinically approved for use in an ampicillin suppository. The caprates, caprylates, and other long-chain saturated fatty acids and their salts can be incorporated into the nanoemulsion process, e.g., into an oil, such as in a pre-emulsion composition. Their hydrophobicity can be enhanced by complexing them, for example, with divalent cations such as those of magnesium, calcium, or zinc, divalent iron, or trivalent iron. Permeabilizers are optional additions to the formulation.

### Gel Capsule

A gel capsule is a soft-shelled capsule, which allows for efficient encapsulation and administration of oil-based drug formulations. A gel capsule may also be referred to as a ‘softgel capsule’ or a ‘caplet’.

Gel capsules may be easier to swallow, avoid dust handling issues, and have increased stability compared to other dosage forms. Gel capsules may be filled with a liquid, such as oils and/or lipid-soluble active ingredients such as pharmaceuticals, veterinary products, foods, and dietary supplements. Soft gel capsules provide an exemplary route for encapsulation and administration of the oil containing DHM and excipients. Gel capsules may be produced from animal sources (e.g., gelatin), algal sources, vegetable sources (e.g., hypromellose (hydroxylpropyl methycellulose, HPMC)), or synthetic sources (e.g., polyvinyl alcohol (PVA), polyethylene glycol (PEG)). Additional examples of materials for producing gel capsules include a polysaccharide, a sulfated polysaccharide, a carrageenan, cellulose, a cellulose derivative, starch, a starch derivative, pullulan, and polyvinyl alcohol (PVA) copolymer. For example, the capsule or gel capsule may be of or include material of algal origin; i.e., the material of which the wall of the capsule is formed may be of or include material of algal origin or derived from an algal material. These and combinations of these and other materials may be used to form a gel capsule (or a capsule). The gel capsules may be filled with an oil containing DHM and an emulsifier (surfactant) and/or other excipients.

The material of which the gel capsule or capsule is formed may be selected to not dissolve or solubilize at low pH (e.g., pH of at most (i.e., less than or equal to) 4.8, 4.5, 4, 3.5, 3.2, 3, 2.7, 2.5, 2.3, 2, 1.8, 1.5, or 1), such as found in the acidic environment of the stomach.

The material of which the gel capsule or capsule is formed may be selected to dissolve or solubilize at near neutral, neutral, or greater than neutral (alkaline) pH (e.g., pH of at least (i.e., greater than or equal to) 5, 5.3, 5.5, 5.8, 6, 6.2, 6.5, 6.7, 7, 7.2, or 7.5, such as found in the intestine. The material of which the gel capsule or capsule is formed may be selected to not dissolve or solubilize in hydrophobic, lipophilic, and/or nonpolar liquids, such as an oil. The material of which the gel capsule or capsule is formed may be selected to dissolve or solubilize in hydrophilic and/or polar liquids, such as water or an aqueous solution. The material of which the gel capsule or capsule is formed may be selected to alter or control the dissolution or solubilization of the gel capsule or capsule, e.g., to alter the rate at and duration of time over which the gel capsule or capsule dissolves or solubilizes to release its contents, e.g., a pre-emulsion composition including a drug (e.g., DHM).

### Enteric Coating

Enteric coatings can be polymers, such as cellulosic compounds, that are applied to the outside of tablets or gel capsules and provide an additional barrier to modify the release characteristics of the contents therein.

Examples of enteric polymers for use on container coatings include shellac, cellulose acetate trimellitate (CAT), various hydroxypropyl cellulose polymers (i.e., HPMC, HPMCP, HPMCAS), and phthalates such as cellulose acetate phthalate (CAP) and polyvinyl acetate phthalate (PVAP). Pros and cons exist for each polymer. Shellac, a natural product derived from an insect secretion, may be subject to inconsistent supply and unacceptable variations in quality.

Cellulose acetate trimellitate may require the potentially undesirable addition of ammonium hydroxide (Wu et al., U.S. Pat. 5,851,579). Hydroxypropylcellulose (HPC) polymers may be unstable upon longer term storage, particularly under conditions of high humidity. Further examples of polymers used to achieve enteric properties in container coatings include anionic polymethacrylates (copolymers of methacrylic acid and either methyl methacrylate or ethyl acrylate) (EUDRAGIT®) such as EUDRAGIT® L 30 D-55 (Methacrylic Acid Copolymer Dispersion, NF), which is soluble at a pH above about 5.5.[44]

The material of which the enteric coating is formed may be selected to not dissolve or solubilize at low pH (e.g., pH of at most (i.e., less than or equal to) 4.8, 4.5, 4, 3.5, 3.2, 3, 2.7, 2.5, 2.3, 2, 1.8, 1.5, or 1), such as found in the acidic environment of the stomach. The material of which the enteric coating is formed may be selected to dissolve or solubilize at near neutral, neutral, or greater than neutral (alkaline) pH (e.g., pH of at least (i.e., greater than or equal to) 5, 5.3, 5.5, 5.8, 6, 6.2, 6.5, 6.7, 7, 7.2, or 7.5, such as found in the intestine. The material of which the enteric coating is formed may be selected to not dissolve or solubilize in hydrophobic, lipophilic, and/or nonpolar liquids, such as an oil. The material of which the enteric coating is formed may be selected to dissolve or solubilize in hydrophilic and/or polar liquids, such as water or an aqueous solution. The material of which the enteric coating is formed may be selected to alter or control the dissolution or solubilization of a gel capsule or capsule that it coats, e.g., to alter the rate at and duration of time over which the gel capsule or capsule dissolves or solubilizes to release its contents, e.g., a pre-emulsion composition including a drug (e.g., DHM).

In some embodiments, DHM constitutes at least 0.01 wt %, 0.03 wt %, 0.1 wt %, 0.2 wt %, 0.5 wt %, 1 wt %, 2 wt %, 5 wt %, 10 wt %, 15 wt %, 20 wt %, 30 wt %, 40 wt %, 50 wt %, 60 wt %, 70 wt %, 80 wt %, 90 wt %, or 99 wt % DHM relative to all other excipients in the pre-emulsion composition. In some embodiments, DHM constitutes at most 0.03 wt %, 0.1 wt %, 0.2 wt %, 0.5 wt %, 1 wt %, 2 wt %, 5 wt %, 10 wt %, 15 wt %, 20 wt %, 30 wt %, 40 wt %, 50 wt %, 60 wt %, 70 wt %, 80 wt %, 90 wt %, 99 wt %, or 99.5 wt % DHM relative to all other excipients in the pre-emulsion composition. In some embodiments, the amount of hydrophilic surfactant in the pre-emulsion composition may be from 0.003 to 50 wt %, from 0.01 to 50 wt %, from 0.01 to 10 wt %, from 0.03 to 3 wt %, from 1 to 10 wt %, or from 3 to 7 wt %. In some embodiments, the amount of co-surfactant in the pre-emulsion composition may be from 0.1 to 50 wt %. For example, the co-surfactant may be present in a ratio relative to the emulsion of 0:1 to 2:1, 0:1 to 1.3:1, or 0.5:1 to 1.3:1. In some embodiments, the amount of permeabilizer in the pre-emulsion composition may be from 0.0001 to 1 wt %, from 0.0001 to 0.1 wt %, from 0.0005 to 0.05 wt %, from 0.001 to 0.02 wt %, or from 0.002 to 0.01 wt %.

In some embodiments, the concentration of all other (non-DHM) components in the pre-emulsion composition may range from 0.001 wt % to 0.01 wt %, or from 0.01 wt % to 0.1 wt %, or from 0.1 wt % to 1 wt %, or from 1 wt % to 10 wt %, or from 10 wt % to 99.9 wt %, depending on the desired release profile, the pharmacological activity and toxicity of the therapeutic compound, and other considerations.

### Nano Emulsion Process

25-30% of pharmaceutical compounds in early development have been estimated to have low solubility and/or bioavailability, reducing the potential efficacy of the drug and often requiring larger doses to achieve therapeutic effects. Such larger doses can have detrimental side effects, and if larger doses must be administered to achieve a therapeutic effect, the therapeutic window (the difference in the dosage that results in a toxic or detrimental effect and the dosage that results in a therapeutic effect) can be narrowed. Because many of these drugs have the potential to be both safe and efficacious, the delivery of these compounds should be improved.

The present invention pertains to emulsification and nanoemulsification processes for forming compositions including liquid dispersions of DHM, a flavonoid, and excipients for the purpose of improving the delivery of flavonoids in animals and humans and improving bioavailability. In an embodiment of the present process, DHM, a surfactant, and a permeabilizer are dissolved or dispersed in an oil phase and the solution filled into a gel capsule that is then sealed and coated with an enteric coating for effective oral delivery. For example, the capsule or gel capsule may be of or include material of algal origin; i.e., the material of which the wall of the capsule is formed may be of or include material of algal origin or derived from an algal material.

Nanoemulsions are oil-in-water emulsions, the oil globules of which have a very fine particle size, e.g., a number-average size of less than or equal to 400 nm. They can be produced by high-energy and high-shear mechanical fragmentation of an oily phase in an aqueous phase in the presence of a surfactant. In the case of nanoemulsions, the small size of the oily globules can be produced by passage through a high-pressure homogenizer. The small size of the globules can render nanoemulsions transparent and exhibiting a novel texture. However, there is the possibility of degradation or instability under these high-energy processes. Low-energy processes can also be used to produce nanoemulsions; they can be grouped under spontaneous emulsification or solvent diffusion, and phase inversion methods.[3-13] Low-energy processes can use changes in temperature or oil/water composition to drive spontaneous emulsification.

Phase inversion methods can be classified as phase inversion temperature (PIT) or phase inversion composition (PIC) methods. As an example of PIT, emulsions stabilized by polyethoxylated (PEO) nonionic surfactants can undergo a phase inversion following a variation of temperature (Shinoda and Saito (1968, 1969)). A so-called transitional phase inversion occurs, when, at a fixed composition, the relative affinity of the surfactant for the different phases is changed and controlled by the temperature. As a result, oil-in-water (o/w) macro-emulsion undergoes a phase inversion to a water-in-oil (w/o) one during a temperature increase, and vice versa. Within the transitional region between both macro-emulsions, for the temperatures at which the nonionic surfactants show very close affinities for the two immiscible phases, the ternary system shows a bicontinuous structure. Characterization of the emulsion inversion and the PIT can be done by measurement of the emulsion electrical conductivity. However, the emulsion phase inversion does not occur for all nonionic polyethoxylated surfactants: even if ethoxy (OE) groups are present in the surfactant molecules, some ethoxylated or polyethoxylated nonionic surfactants, for which the headgroup is either too short or too long, or, vice versa, for which the oily chain is too long or too short, are not sensitive enough to the temperature change to induce a phase inversion. That is, although nonionic polyethoxylated surfactants may enable the emulsion inversion, the affinities of the surfactant for the aqueous and oily bulk phases must be properly balanced. The PIT method includes suddenly breaking up a microemulsion network by performing, at the PIT, a rapid cooling. This stage is an irreversible process, because it leads to the generation of kinetically-stable oil droplets, i.e., the oil-in-water nano-emulsions. The stability results from steric stabilization preventing the droplets flocculating and coalescing. The destabilization of such an emulsion is governed by the inter-droplet oil diffusion (Ostwald ripening).[3]

The PIT process can be more difficult to employ industrially compared to PIC.[10, 11] PIC allows for the formation of nanoemulsions simply through a change in the hydration level of the system. The starting point of the PIC method is a water-in-oil micellar phase, which is an equilibrium phase of the ternary system, in which the surfactant is already at the water/oil interfaces with a curvature turned toward water, or is a surfactant-in-oil phase, with no water or minimal water present, in which inverse micelles form (so that the curvature of the inverse micelles is turned toward the hydrophilic head of the surfactant in the interior of the inverse micelle). The method then proceeds with a large addition of water, which causes an inversion of the spontaneous curvature of the surfactant film, now turned toward the oil. It is found that for certain compositions and with some constraints on the process a homogeneous metastable emulsion, with emulsion droplet diameters in the 100 nm range, can be obtained. Homogeneous can mean that the emulsion droplets may be approximately evenly dispersed. Furthermore, the emulsion droplets may be of similar size.

That is, in the PIC process, phase inversion takes place. That is, the system starts as pure oil (or oil with drug (e.g., DHM), surfactant, and/or other oil-soluble materials dissolved or dispersed in it) and then can go to oil with a small amount of water, or starts as oil (or oil with drug (e.g., DHM), surfactant, and/or other oil-soluble materials dissolved or dispersed in it) with a small amount of water; the system of pure oil or oil with a small amount of water then goes to being predominantly water with oil emulsion drops dispersed therein. That is, the process of going from the organic being the external phase to water being the external phase, is termed phase inversion.

The PIC process can be applied by dissolving or dispersing the drug (e.g., DHM) and the surfactant (emulsifier) and, optionally, co-active(s), permeabilizer(s), and/or other ingredients, in a hydrophobic liquid (an oil) to form a pre-emulsion solution, which can be thermodynamically stable. This pre-emulsion solution can then be stored, sequestered, and/or encapsulated for an indefinite (i.e., a prolonged) period of time. For example, if the pre-emulsion solution is loaded into a capsule formed from gelatin, polyethylene glycol (PEG), or another hydrophilic material, then the capsule will not be dissolved or solubilized (“attacked”) by the oil, so that the capsule can contain the oil (and the drug (e.g., DHM) and other ingredients dissolved or dispersed therein) for an indefinite (i.e., a prolonged) period of time. When the nanoemulsion is to be formed, the pre-emulsion solution is then mixed with water or an aqueous solution. For example, if the capsule is formed of a water-soluble material, such as gelatin, polyethylene glycol, hydroxypropyl methylcellulose acetate succinate (HPMCAS), or another water-soluble cellulose derivative, and the capsule is immersed in water or an aqueous solution, then the capsule (i.e., the wall of the capsule) can dissolve or solubilize, releasing its contents, the pre-emulsion solution, into the water or an aqueous solution. As described above, a homogeneous metastable nanoemulsion, having surfactant-stabilized oil-in-water droplets of a diameter on the order of 100 nm, with the drug (e.g., DHM) in the oil within the droplets, is then rapidly formed. Homogeneous can mean that the droplets may be approximately evenly dispersed. Furthermore, the droplets may be of similar size. For example, the capsule or gel capsule may be of or include material of algal origin; i.e., the material of which the wall of the capsule is formed may be of or include material of algal origin or derived from an algal material.

Transparent microemulsions are known in the art. In contrast to nanoemulsions, microemulsions are not, strictly speaking, emulsions. Whereas nanoemulsions and emulsions are kinetically trapped states, microemulsions are a thermodynamically stable equilibrium phase. Microemulsions are transparent solutions of micelles swollen by oil, which oil is generally a very-short-chain oil (e.g. hexane or decane) and is solubilized by virtue of the joint presence of a significant amount of surfactants and of cosurfactants which form the micelles. The size of the swollen micelles is very small owing to the small amount of oil which they can solubilize. This very small size of the micelles is the cause of their transparency, as with nanoemulsions. However, in contrast to nanoemulsions, microemulsions are thermodynamically stable, and spontaneously formed by mixing the constituents, without contributing mechanical energy other than simple stirring. The major disadvantages of microemulsions are related to their necessarily high proportion of surfactants, leading to potential health and comfort issues. Furthermore, their formulation range is generally narrow and their temperature stability limited.

The use of nano-emulsification technology in the preparation of dosage forms to more effectively deliver DHM and other flavonoids can be advantageous. Nano-emulsification is scalable, that is, it can be implemented at lab, pilot plant, and manufacturing for mass production scales. Nano-emulsification allows for DHM to be formulated with a variety of excipients into an oil or gel that is placed into a capsule to enable bioavailability, efficacy, and minimal cost as needed for a desired administration route. Nanoemulsions can be transparent and DHM in a nanoemulsion can show improved stability relative to free DHM, and nanoemulsions, once formed, can be relatively insensitive to external conditions such as temperature and the chemical environment, compared, for example, to microemulsions.[14] For example, the capsule or gel capsule may be of or include material of algal origin; i.e., the material of which the wall of the capsule is formed may be of or include material of algal origin or derived from an algal material.

### Administration

The resulting formulations of the present invention are useful for delivery in animals and humans and may be administered by oral ingestion. In vivo stability of the present formulation may vary according to the physiological environment to which it is exposed and the excipients and gel capsules used. Therefore, the necessity for or frequency of re-administration may be different for various formulations.

The formulations of embodiments of the present invention may be provided in a variety of ways, for example, in a soft gel capsule dosage form. Additional components that would not significantly prohibit the nanoemulsification process may be added to the formulation prior to formulation of pre-emulsion composition or the nanoemulsion. That is, such additional components should still allow for formulation using the nano-emulsification process.

The resulting formulations of embodiments of the present invention are useful and suitable for delivery in patients, such as humans, animals, and non-human animals, and may be administered by a variety of methods. For the purpose of this application, unless otherwise specified, the term “animal” can be considered to include both non-human animals and humans. Such methods include, by way of example and without limitation, oral, rectal, urethral, or vaginal dosage administration. Such methods of administration and others contemplated within the scope of the present invention are known to the skilled artisan. In vivo stability of the present formulation may vary according to the physiological environment to which it is exposed and the excipients and matrix material used. Therefore, the necessity for or frequency of readministration may be different for various formulations.

For oral administration, the formulation may be in the form of, for example, a gel capsule, such as a soft gel capsule (softgel capsule). For rectal, urethral, or vaginal administration, the formulation may be in the form of for example, a gel capsule suppository for release of compound into the intestines, sigmoid flexure, rectum, urethra, or vagina. For example, the capsule or gel capsule may be of or include material of algal origin; i.e., the material of which the wall of the capsule is formed may be of or include material of algal origin or derived from an algal material. It is contemplated that either one or a combination of long-acting, sustained release, controlled release, or slow release dosage forms may be used in the present invention. For example, this may be achieved by modifying the enteric coating on the gel capsule. The course and duration of administration of and the dosage requirements for the formulation of the present invention may vary according to the subject being treated, the formulation used, the method of administration used, the severity of the condition being treated, the co-administration of other drugs, and other factors.

### Dissolution/Kinetics

In an embodiment, a capsule or softgel capsule that contains a pre-emulsion solution, in which the DHM is dissolved or dispersed, is formed of a material (i.e., the wall of the capsule or softgel capsule is formed of a material) that does not dissolve in and/or is not solubilized by an aqueous solution having a pH of at most (i.e., less than or equal to) 4.8, 4.5, 4, 3.5, 3.2, 3, 2.7, 2.5, 2.3, 2, 1.8, 1.5, or 1. The chyme that is expelled by the stomach, through the pyloric valve, has a pH of approximately 2. Gastric juices (having a pH of from 1 to 3.5) lead to material in the stomach having a pH in the range of from 1 to 3.5, and this low pH in the stomach and the enzymes active in the stomach at this low pH may result in degradation of DHM and quenching of DHM activity.

In an embodiment, a capsule or softgel capsule that contains a pre-emulsion solution, in which the DHM is dissolved or dispersed, is formed of a material (i.e., the wall of the capsule or softgel capsule is formed of a material) that dissolves in and/or is solubilized by water (pH of 7) and/or an aqueous solution having a pH of at least (i.e., greater than or equal to) 5, 5.3, 5.5, 5.8, 6, 6.2, 6.5, 6.7, 7, 7.2, or 7.5. Bile released into the duodenum and/or pancreatic secretions of sodium bicarbonate increase the pH of the chyme. For example, the pH of chyme, material in the intestine (bowel), and intestinal fluid can range from 5.5 to 7.5, for example, can be about 7. The mucosal tissue of the small intestines can have a higher pH, for example, a pH of about 8.5. The dissolution and/or solubilization of the capsule or the softgel capsule (i.e., the wall of the capsule or the softgel capsule) in the intestine, for example, the small intestine, can result in the pre-emulsion solution that contains the drug (e.g., DHM) dissolved or dispersed therein being released into the intestine. As described above, a metastable nanoemulsion, having surfactant-stabilized oil-in-water droplets of a diameter on the order of 100 nm, with the drug (e.g., DHM) in the oil within the droplets, is then rapidly formed in the aqueous environment of the intestine. This metastable nanoemulsion can be described as homogeneous, in the sense that the droplets may be approximately evenly dispersed. Furthermore, the droplets may be of similar size. The drug (e.g., DHM) can then be absorbed by the wall or lining of the intestine, for example, the wall or lining of the small intestine, and into the blood. For example, the drug (e.g., DHM) may diffuse out of a droplet and into the intestinal fluid, and the drug (e.g., DHM) may then be absorbed from the intestinal fluid by the wall or lining of the intestine and into the blood. As an alternative example, a droplet may contact the wall or lining of the small intestine, so that the drug (e.g., DHM) may diffuse directly from the interior of the droplet into the wall or lining of the intestine (i.e., be absorbed by the wall or lining of the intestine), so that the drug (e.g., DHM) then enters the bloodstream.

For example, hydroxypropyl methyl cellulose acetate succinate (HPMCAS) is insoluble in an aqueous solution of acidic (low) pH, but is soluble in an aqueous solution of neutral or alkaline (high) pH. Therefore, a capsule or a softgel capsule (i.e., the wall of the capsule or the softgel capsule) that is formed of HPMCAS can remain intact and retain the pre-emulsion solution at an acidic (low) pH, e.g., a pH of 3.5 or less, but dissolve in or be solubilized by and release the pre-emulsion solution at a neutral or alkaline (high) pH, e.g., a pH of 7 or greater, with the homogeneous metastable nanoemulsion, having surfactant-stabilized oil-in-water droplets of a diameter on the order of 100 nm, with the drug (e.g., DHM) in the oil within the droplets, then being rapidly formed in the aqueous environment in which the capsule or softgel capsule dissolves or is solubilized. Homogeneous can mean that the droplets may be approximately evenly dispersed. Furthermore, the droplets may be of similar size.

A pH buffering agent can be included in the material of which the wall of the capsule or softgel capsule is formed.

Inclusion of an acidic component in the material (e.g., HPMCAS) forming the wall of the capsule or softgel capsule, such as an acidic pH buffering agent (i.e., a buffering agent that maintains an acidic pH, a pH of less than 7), e.g., citric acid or a citrate salt (e.g., a sodium citrate, a potassium citrate, calcium citrate, and/or combinations), can stabilize the wall of the capsule or softgel capsule, so that the wall of the capsule or softgel capsule is not dissolved or solubilized by an aqueous solution or so that the dissolution or solubilization of the wall of the capsule or softgel capsule by the aqueous solution is delayed.

For example, the capsule or gel capsule may be of or include material of algal origin; i.e., the material of which the wall of the capsule is formed may be of or include material of algal origin or derived from an algal material.

An enteric coating of the capsule, softgel capsule, or gel capsule can control, moderate, or delay the dissolution of the wall of the capsule, softgel capsule, or gel capsule in water or an aqueous solution. Examples of polymers used to achieve enteric properties in container coatings, e.g., to form an enteric coating on the exterior of a capsule, softgel capsule, or gel capsule, include anionic polymethacrylates (copolymers of methacrylic acid and either methyl methacrylate or ethyl acrylate) (EUDRAGIT®), such as EUDRAGIT® L 30 D-55 (Methacrylic Acid Copolymer Dispersion, NF), which is soluble at a pH above about 5.5.[44] Thus, an enteric coating, on the exterior of the wall of a capsule, formed of such a methacrylic acid copolymer can prevent dissolution of the capsule in the acidic environment of the stomach, e.g. having a pH from 1 to 3.5, e.g., about 2, even if the wall of the capsule itself would dissolve in or be solubilized by the acidic environment of the stomach. Then, once the capsule passes from the stomach into the intestine, the more neutral environment of the intestine, e.g., having a pH from 5.5 to 7.5, e.g., about 7, can cause such an enteric coating to dissolve or be solubilized, exposing the wall of the capsule to the environment of the intestine. The wall of the capsule can be formed of a material that dissolves in or is solubilized by the neutral or nearly neutral pH environment of the intestine (or that dissolves in or is solubilized by an aqueous solution of any or nearly any pH), so that the wall of the capsule then dissolves or is solubilized, releasing the pre-emulsion solution into the intestine. A homogeneous metastable nanoemulsion, having surfactant-stabilized oil-in-water droplets of a diameter on the order of 100 nm, with the drug (e.g., DHM) in the oil within or at the droplet boundary layer (that may include emulsifier (surfactant) molecules) of the droplets, can then be rapidly formed in the aqueous environment of the intestine, with the drug (e.g., DHM) then being absorbed by the lining of the intestine and into the blood stream. Homogeneous can mean that the droplets may be approximately evenly dispersed. Furthermore, the droplets may be of similar size.

Moderate solubility in water of the wall of the capsule or softgel capsule can allow the wall to dissolve in or be solubilized in the body of an organism and release the pre-emulsion solution, so that a homogeneous metastable nanoemulsion, having surfactant-stabilized oil-in-water droplets of a diameter on the order of 100 nm, with the drug (e.g., DHM) in the oil within the droplets, is then rapidly formed in the aqueous environment of the body, and so that the drug (e.g., DHM) is then absorbed by the body. Homogeneous can mean that the droplets may be approximately evenly dispersed. Furthermore, the droplets may be of similar size. The capsule or softgel capsule wall material can be selected, so that it is moderately soluble (e.g., from 0.01 g/100 mL to 3 g/100 mL, or from 0.1 g/100 mL to 1 g/100 mL) in water. Thus, following ingestion, the capsule, softgel capsule, or gel capsule can dissolve or be solubilized within the stomach (if the material of which the wall of the capsule is formed dissolves in or is solubilized by the low pH environment of the stomach) or intestine (if the material of which the wall of the capsule is formed dissolves in or is solubilized by the neutral (pH=7) or close to neutral environment of the intestine), releasing the oil contents within the capsule and generating the nano-emulsion (an oil-in-water (o/w) emulsion, with the drug (e.g., DHM) within the oil) spontaneously. The nano-emulsion micelles or oil droplets rapidly become widely separated from each other (i.e.., they become dilute) in the aqueous environment of the stomach or intestines. Therefore, coalescence and/or Ostwald ripening of the nano-emulsion micelles or oil droplets does not substantially occur. The large surface area to volume ratio of the nano-emulsion micelles or oil droplets results in a large rate of release (enhanced release kinetics) of the drug (e.g., DHM) from the interior of the nano-emulsion micelles or oil droplets (or from the boundary layer of the nano-emulsion micelles or oil droplets, which can include the emulsifier (surfactant molecules)) into the aqueous environment of the stomach or intestines. The drug (e.g., DHM) can then diffuse or otherwise migrate to the lining of the stomach or the intestines for absorption there into the bloodstream. Also, the nano-emulsion micelles or oil droplets can directly contact the lining of the stomach or intestines, so that the drug (e.g., DHM) diffuses directly from the interior of the micelles or oil droplets to and across the lining of the stomach or intestines and into the bloodstream.

If the desired release is in the stomach, then the capsules with the DHM, excipients and spontaneously emulsifying surfactant phase will release those contents, so that those contents spontaneously disperse and form emulsion droplets of less than 400 nm at 37° C. in simulated gastric fluid. Emulsion size is determined using dynamic light scattering in an instrument such as a Malvern Zeta Sizer. Size is determined from the peak size when the distribution is analyzed using a cumulants deconvolution program, such as provided by Malvern. If the desired release in the intestines, and is effected by an enteric coating, then the capsules with the DHM, excipients and spontaneously emulsifying surfactant phase release those contents, so that those contents spontaneously disperse and form emulsion droplets of less than 400 nm at 37° C. in simulated intestinal fluid. The simulated intestinal fluid will be a fasted state fluid composition. Emulsion size is determined using dynamic light scattering in an instrument such as a Malvern Zeta Sizer. Size is determined from the peak size when the distribution is analyzed using a cumulants deconvolution program, such as provided by Malvern.

The dissolution and release kinetics of DHM are studied under three separate conditions and the protocols are described as follows:[45]

Release Kinetics in Vitro: Simulated gastric fluid (FaSSGF (Fasted-State Simulated Gastric Fluid)) and intestinal fluids (FaSSIF (Fasted-State Simulated Intestinal Fluid) and FeSSIF (Fed-State Simulated Intestinal Fluid)) are prepared according to the manufacturer's instructions. Each formulation is evaluated in triplicate with a release medium swap assay. Additionally, dissolution tests are also performed with DHM-containing gel capsules with the appropriate controls.

Release under Gastric Conditions: DHM-containing gel capsules are suspended in and then dissolve in prewarmed FaSSGF (37° C.) and release the pre-emulsion composition and form the a homogeneous metastable nanoemulsion having the surfactant-stabilized oil-in-water droplets of a diameter on the order of 100 nm, with the drug, e.g., DHM, dissolved or dispersed in the oil within the droplets to achieve a drug concentration of ˜75 μg/mL. Homogeneous can mean that the droplets may be approximately evenly dispersed. Furthermore, the droplets may be of similar size. The samples are incubated at 37° C. (NesLab RTE-111 bath circulator, Thermo Fisher Scientific, Waltham, MA) for 30 min without agitation to mimic physiological gastric conditions and transition time in the stomach. Aliquots are taken at 1, 5, 10, 15, 20, and 30 min. To analyze the free DHM concentration, each aliquot is centrifuged at 28000 g for 5 min to pellet suspended particles. The supernatant is diluted further with FaSSGF to fall within the calibration range, and DHM concentration is determined with a UV-Vis spectrometer at 491 nm. The gel capsules used for the test of release under gastric conditions are made with a wall material that dissolves in or is solubilized by the acidic, low pH (-1.6) FaSSGF. For example, the capsule or gel capsule may be of or include material of algal origin; i.e., the material of which the wall of the capsule is formed may be of or include material of algal origin or derived from an algal material.

Release under Intestinal Conditions: DHM-containing gel capsules are suspended in, but do not dissolve in, prewarmed FaSSGF (37° C.). After passing through the 30 min FaSSGF protocol, the solutions are diluted with 1.1×FaSSIF (pH 6.5) or FeSSIF (pH 5.8). The DHM-containing gel capsules then dissolve and release the pre-emulsion composition and form the a homogeneous metastable nanoemulsion having the surfactant-stabilized oil-in-water droplets of a diameter on the order of 100 nm, with the drug, e.g., DHM, dissolved or dispersed in the oil within the droplets. Homogeneous can mean that the droplets may be approximately evenly dispersed. Furthermore, the droplets may be of similar size. This can result in a final DHM concentration lower than its solubility limit in both buffers. Aliquots are taken at 15, 30, 45, 60, 120, 240, and 360 min after the pH shift and centrifuged at 28000 g for 10 min. The DHM concentration in the supernatant is analyzed with a UV-Vis spectrometer at 491 nm and calculated based on a calibration curve. The gel capsules used for the test of release under gastric conditions are made with a wall material that does not dissolve in and is not solubilized by the acidic, low pH (˜1.6) FaSSGF, but does dissolve in or become solubilized by the mildly acidic, nearly neutral, or neutral solution formed after addition of the FaSSIF or FeSSIF, for example, does dissolve or become solubilized at a pH of at least 5 or 5.5. For example, the capsule or gel capsule may be of or include material of algal origin; i.e., the material of which the wall of the capsule is formed may be of or include material of algal origin or derived from an algal material.

For example, the dissolution kinetics of DHM in a nanoemulsion according to the present invention in in vitro dissolution tests in simulated fasted state fluid are increased by 5%, 10%, 15%, 20%, 30%, 40%, 50%, 60%, 100%, 250%, 500%, or 1000% after 15 minutes over that of pure DHM.

For example, the dissolution kinetics of DHM in a nanoemulsion according to the present invention in in vitro dissolution tests in simulated fed state fluid are increased by 5%, 10%, 15%, 20%, 30%, 40%, 50%, 60%, 100%, 250%, 500%, or 1000% after 15 minutes over that of pure DHM. For example, the dissolution kinetics of DHM in a nanoemulsion according to the present invention in in vitro dissolution tests in simulated fasted state fluid are increased by 5%, 10%, 15%, 20%, 30%, 40%, 50%, 60%, 100%, 250%, 500%, or 1000% after 30 minutes over that of pure DHM.

For example, the dissolution kinetics of DHM in a nanoemulsion according to the present invention in in vitro dissolution tests in simulated fed state fluid are increased by 5%, 10%, 15%, 20%, 30%, 40%, 50%, 60%, 100%, 250%, 500%, or 1000% after 30 minutes over that of pure DHM.

### Animal PK Studies

DHM-containing samples (e.g., gel capsules including a pre-emulsion composition that contains DHM according to the present invention) can be administered (e.g., orally) to an animal (e.g., a rat or a mouse) at 10 mg DHM/kg body weight, or another dosage in an in vivo study, and a pharmacokinetic study can be carried out to evaluate animal pharmacokinetics. The plasma concentration of DHM can be determined, for example, using Waters Acquity ultra performance liquid chromatography equipped with an electrospray ionization mass spectrometry system (Waters, Milford, Mass.), in accordance with a previous report [46], or an equivalent analytical analysis system.

An animal dosed with a gel capsule containing a pre-emulsion composition that contains DHM according to the present invention can show increased blood maximum concentrations, relative to dosing with pure DHM powder, of 5%, 10%, 15%, 20%, 30%, 40%, 50%, 60%, 100%, 250%, 500%, or 1000%. The area under the curve for 24 hours can be increased by 5%, 10%, 15%, 20%, 30%, 40%, 50%, 60%, 100%, 250%, 500%, or 1000% over the value associated with dosing with pure DHM powder.

For example, the capsule or gel capsule may be of or include material of algal origin; i.e., the material of which the wall of the capsule is formed may be of or include material of algal origin or derived from an algal material.

### Advantages of the Nanoemulsion Process

In the present invention, the use of nanoemulsion technology in the preparation of dosage forms to more effectively deliver drugs, such as flavonoids, e.g., DHM, has several advantages.

The production of pre-emulsion compositions and their encapsulation in a gel capsule is scalable, e.g., to the lab scale, pilot plant scale, and industrial mass-production scale. Nanoemulsion technology allows for DHM to be formulated with a wide variety of excipients to allow for maximum bioavailability and efficacy for the desired administration route. Because the drug, e.g., DHM, molecules are dissolved in or solubilized (dispersed) in the pre-emulsion composition, e.g., an oil, and, after the pre-emulsion composition is released from the capsule in the body, for example, in the intestine, a homogeneous metastable nanoemulsion is formed that has surfactant-stabilized oil-in-water droplets of a diameter on the order of 100 nm, with the drug, e.g., DHM, dissolved or dispersed in the oil within the droplets, the drug, e.g., DHM, molecules can be maintained in a dissolved, “disordered”, non-aggregated, and/or non-crystalline state that facilitates absorption of the drug, e.g., DHM, by the body, e.g., through the lining of the intestine and into the bloodstream. This can result in high bioavailability and sustained concentrations of the drug, e.g., DHM, in the bloodstream as needed. Homogeneous can mean that the droplets may be approximately evenly dispersed. Furthermore, the droplets may be of similar size.

For example, the capsule or gel capsule may be of or include material of algal origin; i.e., the material of which the wall of the capsule is formed may be of or include material of algal origin or derived from an algal material.

Several nonlimiting Aspects of the invention are set forth below:

Aspect 1. A dihydromyricetin (DHM) pre-emulsion composition, comprising: dihydromyricetin (DHM); and an emulsifier.

Aspect 2. The DHM pre-emulsion composition of Aspect 1, wherein the emulsifier comprises a nonionic surfactant.

Aspect 3. The DHM pre-emulsion composition of any one of Aspects 1 and 2, wherein the emulsifier has a hydrophilic-lipophilic balance (HLB) greater than 7.

Aspect 4. The DHM pre-emulsion composition of any one of Aspects 1 through 3, wherein the emulsifier comprises a polymeric emulsifier.

Aspect 5. The DHM pre-emulsion composition of any one of Aspects 1 through 4, wherein the emulsifier comprises an amphiphilic block copolymer.

Aspect 6. The DHM pre-emulsion composition of any one of Aspects 1 through 5, wherein the emulsifier comprises a copolymer of ethylene oxide.

Aspect 7. The DHM pre-emulsion composition of any one of Aspects 1 through 6, wherein the emulsifier comprises a homopolymer of ethylene oxide.

Aspect 8. The DHM pre-emulsion composition of any one of Aspects 1 through 7, wherein the emulsifier comprises a polyoxyethylene-polyoxypropylene block copolymer.

Aspect 9. The DHM pre-emulsion composition of Aspect 8,
wherein the polyoxyethylene-polyoxypropylene block copolymer has the formula

HO(C₂H₄O)_{A}(C₃H₆O)_{B}(C₂H₄O)_{A}H,
wherein A denotes the number of polyoxyethylene units,
wherein A is from 1 to 100,
wherein B denotes the number of polyoxypropylene units, and
wherein B is from 1 to 100.

Aspect 10. The DHM pre-emulsion composition of any one of Aspects 1 through 9, wherein the emulsifier comprises a polyethoxylated fatty acid.

Aspect 11. The DHM pre-emulsion composition of any one of Aspects 1 through 10, wherein the emulsifier comprises a polyethylene glycol sorbitan fatty acid ester (polysorbate).

Aspect 12. The DHM pre-emulsion composition of any one of Aspects 1 through 11, wherein the emulsifier comprises a polysorbate selected from the group consisting of polyoxyethylene sorbitan monolaurate (Tween 20) , polyoxyethylene sorbitan monopalmitate (Tween 40), polyoxyethylene sorbitan monostearate (Tween 60) , polyoxyethylene sorbitan tristearate (Tween 65), polyoxyethylene sorbitan mono- oleate (Tween 80), and combinations.

Aspect 13. The DHM pre-emulsion composition of any one of Aspects 1 through 12, wherein the emulsifier comprises a polyethoxyethylene sorbitan monoester.

Aspect 14. The DHM pre-emulsion composition of any one of Aspects 1 through 13, wherein the emulsifier comprises a sugar surfactant.

Aspect 15. The DHM pre-emulsion composition of any one of Aspects 1 through 14, wherein the emulsifier comprises a sugar ester.

Aspect 16. The DHM pre-emulsion composition of any one of Aspects 1 through 15, wherein the emulsifier comprises a sugar fatty acid ester.

Aspect 17. The DHM pre-emulsion composition of any one of Aspects 1 through 16, wherein the emulsifier comprises a sugar fatty acid ester selected from the group consisting of sucrose monopalmitate, sucrose monolaurate, sucrose monostearate, sucrose distearate, sucrose monopalmitate, sucrose dipalmitate, sucrose monolaurate, saccharose monolaurate, and combinations.

Aspect 18. The DHM pre-emulsion composition of any one of Aspects 1 through 17, wherein the emulsifier comprises an emulsifier selected from the group consisting of a polyethylene glycol alkyl ether, a polyethylene glycol alkyl phenol, a polyglycerol ester, a polyethoxylated fatty acid diester, a polyethylene glycol fatty acid monoester, a polyethylene glycol fatty acid diester, a polyethylene glycol glycerol fatty acid ester, and combinations.

Aspect 19. The DHM pre-emulsion composition of any one of Aspects 1 through 18, wherein the emulsifier comprises a citric acid ester of a monoglyceride and/or an alcohol oil transester.

Aspect 20. The DHM pre-emulsion composition of any one of Aspects 1 through 19, wherein the emulsifier comprises a polyvinyl alcohol.

Aspect 21. The DHM pre-emulsion composition of any one of Aspects 1 through 20, wherein the emulsifier comprises an alkyl cellulose, an alkyl guar, a hydroxyalkyl guar, and/or derivatives of these.

Aspect 22. The DHM pre-emulsion composition of any one of Aspects 1 through 21, wherein the emulsifier comprises a C₁-C₂ alkyl cellulose, a C₁-C₃ alkyl guar, a C₁-C₃ hydroxyalkyl guar, and/or derivatives of these.

Aspect 23. The DHM pre-emulsion composition of any one of Aspects 1 through 22, wherein the emulsifier comprises a polyvinylpyrrolidone, a polyvinylpyrrolidone copolymer, polyvinylcaprolactam, and/or a polyvinylcaprolactam copolymer.

Aspect 24. The DHM pre-emulsion composition of any one of Aspects 1 through 23, wherein the emulsifier comprises a polyvinyl methyl ether and/or a polyvinyl methyl ether copolymer.

Aspect 25. The DHM pre-emulsion composition of any one of Aspects 1 through 24, wherein the emulsifier comprises an anionic surfactant.

Aspect 26. The DHM pre-emulsion composition of any one of Aspects 1 through 25, wherein the emulsifier comprises an anionic amphiphilic lipid.

Aspect 27. The DHM pre-emulsion composition of any one of Aspects 1 through 26, wherein the emulsifier comprises a polyacrylic acid, a polyacrylic acid copolymer, polyacrylic acid crosslinked with alkyl sucrose, polyacrylic acid crosslinked with allyl pentaerythritol, poly(acrylic acid-co-alkylacrylate), poly(acrylic acid-co-alkylacrylate) crosslinked with alkyl sucrose, poly(acrylic acid-co-alkylacrylate) crosslinked with allyl pentaerythritol, a polyacrylic acid salt (e.g., sodium polyacrylate), and/or a polyacrylic acid copolymer salt.

Aspect 28. The DHM pre-emulsion composition of any one of Aspects 1 through 27, wherein the emulsifier comprises an emulsifier selected from the group consisting of a fatty acid ester, a fatty alcohol ester, a carboxylic acid ester, a glycerol ester, a mixed ester of a fatty acid, a fatty alcohol, a carboxylic acid, and/or glycerol, and combinations.

Aspect 29. The DHM pre-emulsion composition of any one of Aspects 1 through 28, wherein the emulsifier comprises an alkyl ether citrate.

Aspect 30. The DHM pre-emulsion composition of any one of Aspects 1 through 29, wherein the emulsifier comprises an emulsifier selected from the group consisting of an alkenyl succinate, an alkoxylated alkenyl succinate, an alkoxylated glucose alkenyl succinate, an alkoxylated methylglucose alkenyl succinate, and combinations.

Aspect 31. The DHM pre-emulsion composition of any one of Aspects 1 through 30, wherein the emulsifier comprises an emulsifier selected from the group consisting of a phosphoric acid fatty ester, alkaline salts of dicetyl and dimyristyl phosphate, alkaline salts of cholesterol sulfate, alkaline salts of cholesterol phosphate, lipoamino acids and their salts, mono- and disodium acylglutamates, disodium salt of N-stearoyl-L-glutamic acid, sodium salts of phosphatidic acid, phospholipids, and combinations.

Aspect 32. The DHM pre-emulsion composition of any one of Aspects 1 through 31, wherein the emulsifier comprises an alkylsulfonic compound, an alkylsulfonic derivative, and/or combinations.

Aspect 33. The DHM pre-emulsion composition of any one of Aspects 1 through 32, wherein the emulsifier comprises a cationic surfactant.

Aspect 34. The DHM pre-emulsion composition of any one of Aspects 1 through 33, wherein the emulsifier comprises a cationic amphiphilic lipid.

Aspect 35. The DHM pre-emulsion composition of any one of Aspects 1 through 34, wherein the emulsifier comprises an emulsifier selected from the group consisting of a quaternary ammonium salts, a fatty amine, a primary fatty amine, a secondary fatty amine, a tertiary fatty amine, a fatty amine salt, a primary fatty amine salt, a secondary fatty amine salt, a tertiary fatty amine salt, derivatives of these, and combinations.

Aspect 36. The DHM pre-emulsion composition of any one of Aspects 1 through 35, further comprising a co-surfactant.

Aspect 37. The DHM pre-emulsion composition of any one of Aspects 1 through 36, wherein the co-surfactant is selected from the group consisting of a sorbitan fatty acid esters, sorbitan monolaurate, sorbitan monopalmitate, sorbitan tristearate, sorbitan monostearate, sorbitan monooleate, sorbitan trioleate, a phospholipid, egg lecithin, soy lecithin, epikuron, topcithin, leciprime, lecisoy, emulfluid, emulpur, metarin, emultop, lecigran, lecimulthin, hydroxylated lecithin, lysophosphatidylcholine, cardiolipin, sphingomyelin , phosphatidylcholine, phosphatidyl ethanolamine, phosphatidic acid, phosphatidyl glycerol, phosphatidyl serine, an ionic surfactant, sodium stearoyl lactylate, calcium stearoyl lactylate, and combinations.

Aspect 38. The DHM pre-emulsion composition of any one of Aspects 1 through 37, further comprising an oil.

Aspect 39. The DHM pre-emulsion composition of Aspect 38, wherein the oil has a molecular weight of at least 400 Da.

Aspect 40. The DHM pre-emulsion composition of Aspect 38, wherein the oil has a molecular weight of less than 400 Da.

Aspect 41. The DHM pre-emulsion composition of any one of Aspects 38 through 40, wherein the DHM is dispersed in the oil.

Aspect 42. The DHM pre-emulsion composition of any one of Aspects 38 through 40, wherein the DHM is dissolved in the oil.

Aspect 43. The DHM pre-emulsion composition of any one of Aspects 1 through 42, further comprising a permeabilizer.

Aspect 44. The DHM pre-emulsion composition of Aspect 43, wherein the permeabilizer comprises capric acid, a caprate salt, sodium caprate, caprylic acid, a caprylate salt, and/or sodium caprylate.

Aspect 45. The DHM pre-emulsion composition of Aspect 43, wherein the permeabilizer comprises a permeabilizer selected from the group consisting of a fatty acid, a saturated fatty acid, and/or a fatty acid complexed with a cation, such as a metal cation, a metal divalent cation, a magnesium divalent cation, a calcium divalent cation, a zinc divalent cation, an iron divalent cation, a metal trivalent cation, an iron trivalent cation, a fatty acid salt, a fatty acid metallic soap, and combinations.

Aspect 46. The DHM pre-emulsion composition of any one of Aspects 1 through 45, further comprising a coactive.

Aspect 47. The DHM pre-emulsion composition of Aspect 46, wherein the coactive comprises an antioxidant.

Aspect 48. The DHM pre-emulsion composition of any one of Aspects 46 and 47, wherein the coactive comprises an electrolyte and/or a sugar.

Aspect 49. The DHM pre-emulsion composition of Aspect 46, wherein the coactive comprises glutathione.

Aspect 50. The DHM pre-emulsion composition of Aspect 46, wherein the coactive comprises L-cysteine.

Aspect 51. The DHM pre-emulsion composition of Aspect 46, wherein the coactive comprises a coactive selected from the group consisting of N-acetyl cysteine (NAC), Prickly

Pear extract, Milk Thistle, ginger root, vitamin B, vitamin C, vitamin E, and combinations.

Aspect 52. The DHM pre-emulsion composition of any one of Aspects 1 through 51, which disperses to emulsion droplets of diameter of at most 10,000 nanometers when contacted with an excess aqueous phase.

Aspect 53. The DHM pre-emulsion composition of any one of Aspects 1 through 51, which disperses to emulsion droplets of diameter of at most 3000 nanometers when contacted with an excess aqueous phase.

Aspect 54. The DHM pre-emulsion composition of any one of Aspects 1 through 51, which disperses to emulsion droplets of diameter of at most 1000 nanometers when contacted with an excess aqueous phase.

Aspect 55. The DHM pre-emulsion composition of any one of Aspects 1 through 51, which disperses to emulsion droplets of diameter of at most 400 nanometers when contacted with an excess aqueous phase.

Aspect 56. The DHM pre-emulsion composition of any one of Aspects 1 through 51, which disperses to emulsion droplets of diameter of at most 200 nanometers when contacted with an excess aqueous phase.

Aspect 57. A dosage form, comprising:
the dihydromyricetin (DHM) pre-emulsion composition of any one of Aspects 1 through 56; and
a capsule,
wherein the DHM pre-emulsion composition is encapsulated in the capsule.

Aspect 58. The dosage form of Aspect 57, wherein the capsule is a soft gel capsule.

Aspect 59. The dosage form of Aspect 57 or 58, wherein the capsule comprises animal-derived material, such as gelatin and/or collagen.

Aspect 60. The dosage form of Aspect 57 or 58, wherein the capsule comprises plant-derived material.

Aspect 61. The dosage form of Aspect 57 or 58, wherein the capsule comprises synthetically-produced material.

Aspect 62. The dosage form of Aspect 57 or 58, wherein the capsule comprises a polysaccharide, a sulfated polysaccharide, a carrageenan, cellulose, a cellulose derivative, starch, a starch derivative, pullulan, polyvinyl alcohol (PVA), polyvinyl alcohol (PVA) copolymer, polyethylene glycol (PEG), and combinations.

Aspect 63. The dosage form of Aspect 57 or 58, wherein the capsule comprises hydroxypropyl methylcellulose (HPMC).

Aspect 64. The dosage form of Aspect 57 or 58, wherein the capsule comprises hydroxypropyl methyl cellulose acetate succinate (HPMCAS).

Aspect 65. The dosage form of Aspect 57 or 58, wherein the capsule comprises material of algal origin.

Aspect 66. The dosage form of Aspect 57 or 58, wherein the capsule comprises material derived from material of algal origin.

Aspect 67. The dosage form of any one of Aspects 57 through 66, wherein the capsule is not solubilized or dissolved by an aqueous solution having a pH of at most 3.5.

Aspect 68. The dosage form of any one of Aspects 57 through 66, wherein the capsule is not solubilized or dissolved by an aqueous solution having a pH of at most 2.

Aspect 69. The dosage form of any one of Aspects 57 through 68, wherein the capsule is solubilized or dissolved by an aqueous solution having a pH of at least 5.5.

Aspect 70. The dosage form of any one of Aspects 57 through 68, wherein the capsule is solubilized or dissolved by water or an aqueous solution having a pH of at least 7.

Aspect 71. The dosage form of any one of Aspects 57 through 70,
wherein the capsule comprises an exterior surface and
wherein the exterior surface is coated with an enteric coating.

Aspect 72. The dosage form of Aspect 71, wherein the enteric coating is a polymeric coating.

Aspect 73. The dosage form of Aspect 71, wherein the enteric coating is a methacrylate copolymer coating.

Aspect 74. The dosage form of Aspect 71, wherein the enteric coating is a hydroxypropyl methyl cellulose acetate succinate (HPMCAS) coating.

Aspect 75. A dihydromyricetin (DHM) pre-emulsion composition, comprising: dihydromyricetin (DHM);
polyoxyethylene sorbitan monolaurate;
caprylic acid and/or capric acid; and
soybean oil.

Aspect 76. The dihydromyricetin (DHM) pre-emulsion composition of Aspect 75,
wherein the dihydromyricetin (DHM) is at a concentration of from 3 to 30 mg/mL,
wherein the polyoxyethylene sorbitan monolaurate is at a concentration of from 0.3 to 3 mg/mL, and
wherein the caprylic acid and/or capric acid is at a concentration of from 0.02 mg/mL to 0.2 mg/mL
in the soybean oil.

Aspect 77. A dosage form, comprising:
the dihydromyricetin (DHM) pre-emulsion composition of any one of Aspects 75 and 76; and
a softgel capsule,
wherein the DHM pre-emulsion composition is encapsulated in the capsule.

Aspect 78. The dosage form of Aspect 77, wherein a wall of the softgel capsule comprises gelatin.

Aspect 79. A method for forming the dosage form of any one of Aspects 57 through 74, 77, and 78, comprising:
mixing the dihydromyricetin (DHM), the emulsifier, and an oil to dissolve or disperse the DHM and the emulsifier in the oil to form the DHM pre-emulsion composition;
loading the DHM pre-emulsion composition into the capsule; and sealing the capsule.

Aspect 80. A method for administering dihydromyricetin (DHM) to a patient, comprising:
orally administering the dosage form of any one of Aspects 57 through 74, 77, and 78 to the patient,
allowing the capsule to enter the patient's stomach, where the capsule is not dissolved and is not solubilized by gastric juices in the stomach,
allowing the capsule to pass from the stomach to the patient's intestine, where the capsule is dissolved or solubilized by intestinal fluid in the intestine,
allowing the partially or fully dissolved or solubilized capsule to release the DHM pre-emulsion composition into the intestinal fluid,
allowing the released DHM pre-emulsion composition to form a metastable nanoemulsion comprising oil-in-water droplets in the intestinal fluid, and
allowing the DHM to diffuse from the oil-in-water droplets into a wall of the intestine and into the patient's bloodstream, so that the DHM is administered to the patient.

Aspect 81. The method for administering DHM to a patient of Aspect 80, wherein the oil-in-water droplets of the nanoemulsion have a diameter of at most 10,000 nm, 3000 nm, 1000 nm, 400 nm, or 200 nm.

Aspect 82. The method for administering DHM to a patient of any one of Aspects 80 and 81, wherein the patient is a human.

Aspect 83. The method for administering DHM to a patient of any one of Aspects 80 and 81, wherein the patient is a non-human animal.

Aspect 84. The dihydromyricetin (DHM) pre-emulsion composition of any one of Aspects 1 through 56, 75, and 76 or the dosage form according to any one of Aspects 57 through 74, 77, and 78 for use as a medicament.

Aspect 85. The dihydromyricetin (DHM) pre-emulsion composition of any one of Aspects 1 through 56, 75, and 76 or the dosage form according to any one of Aspects 57 through 74, 77, and 78 for use in reducing hangover symptoms.

Aspect 86. The dihydromyricetin (DHM) pre-emulsion composition of any one of Aspects 1 through 56, 75, and 76 or the dosage form according to any one of Aspects 57 through 74, 77, and 78 for use in preventing an alcohol use disorder.

Aspect 87. The dihydromyricetin (DHM) pre-emulsion composition of any one of Aspects 1 through 56, 75, and 76 or the dosage form according to any one of Aspects 57 through 74, 77, and 78 for use in preventing alcoholism.

Aspect 88. The dihydromyricetin (DHM) pre-emulsion composition of any one of Aspects 1 through 56, 75, and 76 or the dosage form according to any one of Aspects 57 through 74, 77, and 78 for use in treating an alcohol use disorder.

Aspect 89. The dihydromyricetin (DHM) pre-emulsion composition of any one of Aspects 1 through 56, 75, and 76 or the dosage form according to any one of Aspects 57 through 74, 77, and 78 for use in treating alcoholism.

Aspect 90. The dihydromyricetin (DHM) pre-emulsion composition of any one of Aspects 1 through 56, 75, and 76 or the dosage form according to any one of Aspects 57 through 74, 77, and 78 for use in treating an alcohol overdose.

Aspect 91. The dihydromyricetin (DHM) pre-emulsion composition of any one of Aspects 1 through 56, 75, and 76 or the dosage form according to any one of Aspects 57 through 74, 77, and 78 for use in increasing antioxidant capacity.

Aspect 92. The dihydromyricetin (DHM) pre-emulsion composition of any one of Aspects 1 through 56, 75, and 76 or the dosage form according to any one of Aspects 57 through 74, 77, and 78 for use in neuroprotection.

Aspect 93. The dihydromyricetin (DHM) pre-emulsion composition of any one of Aspects 1 through 56, 75, and 76 or the dosage form according to any one of Aspects 57 through 74, 77, and 78 for use in preventing Alzheimer's disease.

Aspect 94. The dihydromyricetin (DHM) pre-emulsion composition of any one of Aspects 1 through 56, 75, and 76 or the dosage form according to any one of Aspects 57 through 74, 77, and 78 for use in treating Alzheimer's disease.

Aspect 95. The dihydromyricetin (DHM) pre-emulsion composition of any one of Aspects 1 through 56, 75, and 76 or the dosage form according to any one of Aspects 57 through 74, 77, and 78 for use in inhibiting inflammation.

Aspect 96. The dihydromyricetin (DHM) pre-emulsion composition of any one of Aspects 1 through 56, 75, and 76 or the dosage form according to any one of Aspects 57 through 74, 77, and 78 for use in protection of the kidney.

Aspect 97. The dihydromyricetin (DHM) pre-emulsion composition of any one of Aspects 1 through 56, 75, and 76 or the dosage form according to any one of Aspects 57 through 74, 77, and 78 for use in protection of the liver.

Aspect 98. The dihydromyricetin (DHM) pre-emulsion composition of any one of Aspects 1 through 56, 75, and 76 or the dosage form according to any one of Aspects 57 through 74, 77, and 78 for use in preventing or treating cancer.

Aspect 99. The dihydromyricetin (DHM) pre-emulsion composition of any one of Aspects 1 through 56, 75, and 76 or the dosage form according to any one of Aspects 57 through 74, 77, and 78 for use in ameliorating a metabolic disorder.

Aspect 100. The dihydromyricetin (DHM) pre-emulsion composition of any one of Aspects 1 through 56, 75, and 76 or the dosage form according to any one of Aspects 57 through 74, 77, and 78 for use in preventing diabetes.

Aspect 101. The dihydromyricetin (DHM) pre-emulsion composition of any one of Aspects 1 through 56, 75, and 76 or the dosage form according to any one of Aspects 57 through 74, 77, and 78 for use in treating diabetes.

Aspect 102. The dihydromyricetin (DHM) pre-emulsion composition of any one of Aspects 1 through 56, 75, and 76 or the dosage form according to any one of Aspects 57 through 74, 77, and 78 for use in treating a bacterial infection.

Aspect 103. Use of the dihydromyricetin (DHM) pre-emulsion composition of any one of Aspects 1 through 56, 75, and 76 in the manufacture of a medicament for reducing hangover symptoms.

Aspect 104. Use of the dihydromyricetin (DHM) pre-emulsion composition of any one of Aspects 1 through 56, 75, and 76 in the manufacture of a medicament for preventing an alcohol use disorder, preventing alcoholism, treating an alcohol use disorder, treating alcoholism, and/or treating an alcohol overdose.

Aspect 105. Use of the dihydromyricetin (DHM) pre-emulsion composition of any one of Aspects 1 through 56, 75, and 76 in the manufacture of a medicament for neuroprotection, preventing Alzheimer's disease, and/or treating Alzheimer's disease.

Aspect 106. Use of the dihydromyricetin (DHM) pre-emulsion composition of any one of Aspects 1 through 56, 75, and 76 in the manufacture of a medicament for ameliorating a metabolic disorder, preventing diabetes, and/or treating diabetes.

Aspect 107. Use of the dihydromyricetin (DHM) pre-emulsion composition of any one of Aspects 1 through 56, 75, and 76 in the manufacture of a medicament for increasing antioxidant capacity, inhibiting inflammation, protecting the kidney, protecting the liver, preventing and/or treating cancer, and/or treating a bacterial infection.

### EXAMPLES

The following examples provide a detailed description of particular embodiments of the invention. It is recognized that departures from the disclosed embodiment may be made within the scope of the invention and that obvious modifications will occur to a person skilled in the art. The claims and specification should not be construed to unduly narrow the full scope of protection to which the invention is entitled.

### Example 1

Pure dihydromyricetin (DHM) crystals, Tween 20 (polyoxyethylene sorbitan monolaurate) nonionic surfactant, and caprylic (octanoic) acid or capric (decanoic) acid are mixed together in soybean oil at concentrations of 10, 0.8, and 0.05 mg/mL respectively. This oil mixture is stirred gently for 30 mins to fully dissolve or disperse all the components at a slightly elevated temperature of 40° C. Once dissolved or dispersed, the oil mixture (the pre-emulsion composition) is cooled to room temperature then added to the input stream of softgel encapsulation equipment manufactured by CapPlus Technologies. The encapsulating material is gelatin. The oil is rapidly encapsulated into the softgel capsules and is then sent to secondary drying and dehumidification steps for several hours. Following drying, the capsules are sent for packaging, storage, and distribution.

### Example 2

Pure dihydromyricetin (DHM) crystals, Tween 20 (polyoxyethylene sorbitan monolaurate) nonionic surfactant, and caprylic (octanoic) acid or capric (decanoic) acid are mixed together in soybean oil at concentrations of 10, 0.8, and 0.05 mg/mL respectively. This oil mixture is stirred gently for 30 mins to fully dissolve or disperse all the components at a slightly elevated temperature of 40° C. Once dissolved or dispersed, the oil mixture (the pre-emulsion composition) is cooled to room temperature then added to the input stream of softgel encapsulation equipment manufactured by CapPlus Technologies. The encapsulating material is of algal origin or derived from material of algal origin. The oil is rapidly encapsulated into the softgel capsules and is then sent to secondary drying and dehumidification steps for several hours. Following drying, the capsules are sent for packaging, storage, and distribution.

### Example 3

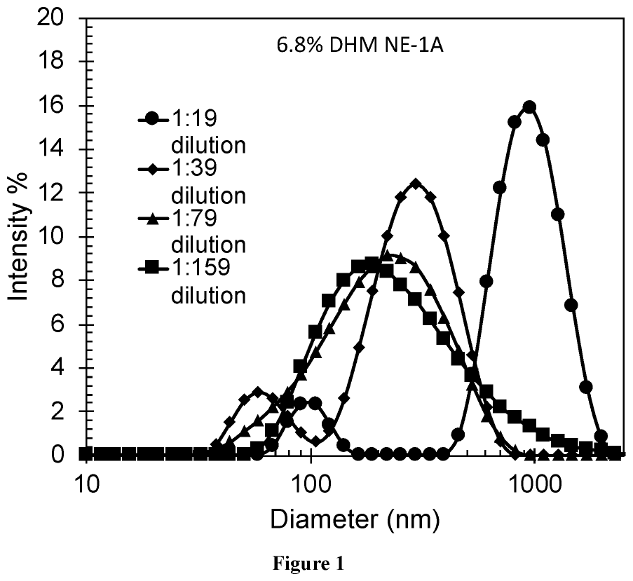

Capmul MCM (2,3-dihydroxypropyl octanoate), Kolliphor EL (polyethoxylated castor oil), and Transcutol (2-(2-ethoxyethoxy)ethanol) are mixed with a mass ratio of Capmul:Kolliphor:Transcutol of 1:2:1 (abbreviation NE-1). Various amounts of pure dihydromyricetin (DHM) crystals and water are added to make the total DHM mass percentage in the mixture from 4.3 wt % to 7.3 wt % and the water mass percentage from 43 wt % to 78 wt %. The mixture is stirred using a magnetic stirring bar at 40° C., and the mixture is observed to determine whether a clear and homogeneous nanoemulsion is produced. The results are summarized in Table 1, where “Y” indicates observation of a homogeneous nanoemulsion and “N” indicates observation of a cloudy suspension. A formulation of 12.6 wt % Capmul MCM, 25 wt % Kolliphor, 12.6 wt % Transcutol, 43 wt % water, and 6.8 wt % DHM is chosen and is defined as NE-1A. NE-1A is a homogenous clear nanoemulsion. FIG. 1 indicates the distribution of droplet sizes for sequential water dilution from dynamic light scattering. 1:19 dilution means a mass ratio of 1 part of NE-1A mixed with 19 parts of water. Sequential dilution is used, so that the 1:39 mixture is made with 1 part of the 1:19 dilution mixture mixed with an additional 1 part of water.

### Example 4

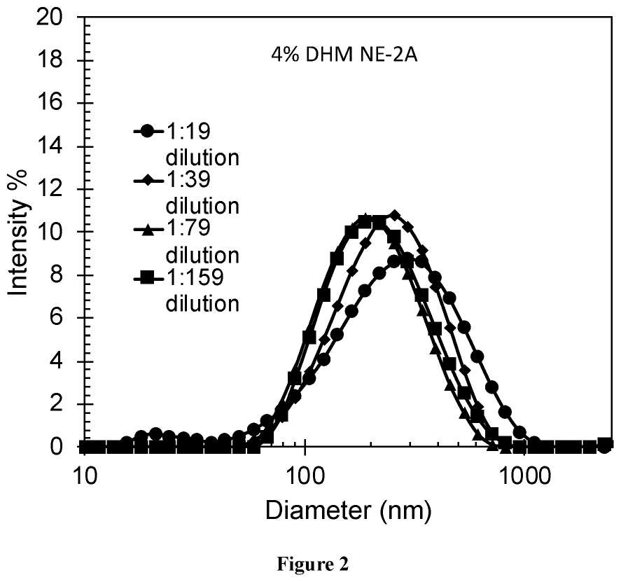

Capmul MCM (2,3-dihydroxypropyl octanoate), Kolliphor EL (polyethoxylated castor oil), and Transcutol (2-(2-ethoxyethoxy)ethanol) are mixed with a mass ratio of Capmul:Kolliphor:Transcutol of 1:2:0.5 (abbreviation NE-2). Various amount of pure dihydromyricetin (DHM) crystals and water are added to make total DHM mass percentage in the mixture from 2.6 wt % to 4.6 wt % and water mass percentage from 52 wt % to 82 wt %. The mixture is stirred using a magnetic stirring bar at 40° C., and the mixture is observed to determine whether a clear and homogeneous nanoemulsion is produced. The results are summarized in Table 2, where “Y” indicates observation of a homogeneous nanoemulsion and “N” indicates observation of a cloudy suspension. A formulation of 12.6 wt % Capmul, 25 wt % Kolliphor, 6.4 wt % Transcutol, 52 wt % water, and 4 wt % DHM is chosen and is defined as NE-2A. NE-2A is a homogenous clear nanoemulsion. FIG. 2 indicates the distribution of droplet sizes for sequential water dilution from dynamic light scattering. 1:19 dilution means a mass ratio of 1 part of NE-2A mixed with 19 parts of water. Sequential dilution is used, so that the 1:39 mixture is made with 1 part of the 1:19 dilution mixture mixed with an additional 1 part of water.

### Example 5

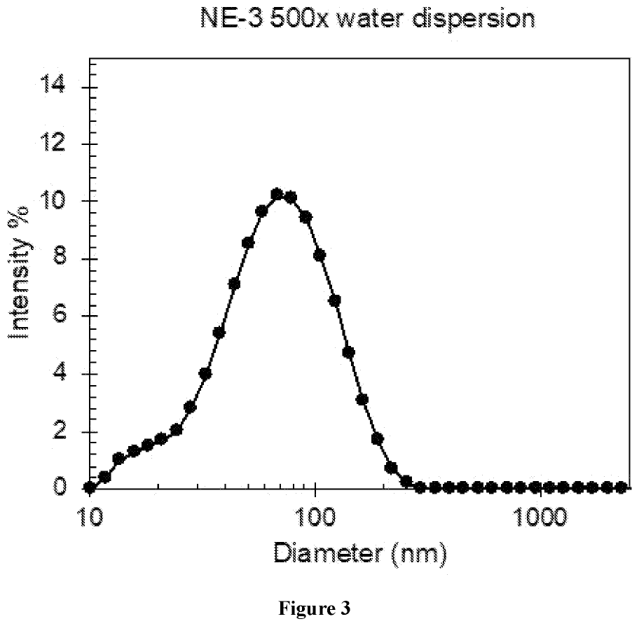

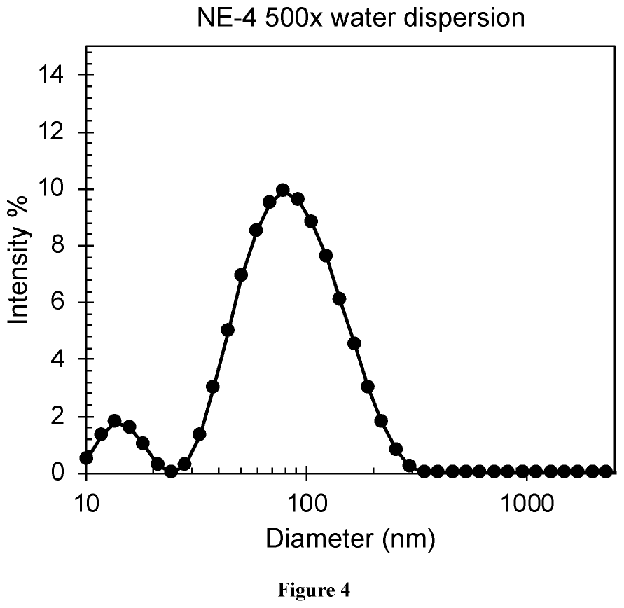

The formulation of this Example is made without water and can be directly loaded into softgel capsules. Capmul MCM (2,3-Dihydroxypropyl octanoate), Kolliphor EL (polyethoxylated castor oil), Transcutol (2-(2-ethoxyethoxy)ethanol), and pure dihydromyricetin (DHM) crystals are mixed at 40° C. with a mass ratio of Capmul:Kolliphor:Transcutol:DHM of 1:2:1:1 (abbreviation NE-3, 20 wt % DHM drug loading). Upon dosage and the mixing of the formulation with water, a nanoemulsion with nanosized droplets is formed. FIG. 3 indicates the distribution of droplet sizes resulting from a 1:499 dilution of the NE-3 formulation with water, i.e., a mass ratio of 1 part of NE-3 mixed with 499 parts of water.

### Example 6

The formulation of this Example is made without water and can be directly loaded into softgel capsules. Capmul MCM (2,3-Dihydroxypropyl octanoate), Kolliphor EL (polyethoxylated castor oil), Transcutol (2-(2-ethoxyethoxy)ethanol), and pure dihydromyricetin (DHM) crystals are mixed at 40° C. with a mass ratio of Capmul:Kolliphor:Transcutol:DHM of 13:26:7:7 (abbreviation NE-4, 13% DHM drug loading). Upon dosage and the mixing of the formulation with water, a nanoemulsion with nanosized droplets is formed. FIG. 4 indicates the distribution of droplet sizes resulting from a 1:499 dilution of the NE-4 formulation with water, i.e., a mass ratio of 1 part of NE-4 mixed with 499 parts of water.

### Example 7A

13 wt % Capmul MCM (2,3-Dihydroxypropyl octanoate), 26 wt % Kolliphor EL (polyethoxylated castor oil), 13 wt % Transcutol (2-(2-ethoxyethoxy)ethanol), 4.3 wt % pure dihydromyricetin (DHM) crystals, and 43.7 wt % water are mixed at 40° C. to make formulation NE-5. 1.4 g of NE-5 is placed into 6-8kD dialysis tubing, which is sealed at both ends. The nanoemulsion is placed inside a dialysis membrane to separate the DHM molecules released into the medium from the nanodroplets, which encapsulate DHM. (Direct centrifugation of the bulk does not result in a perfect separation of the released DHM from the nanodroplets. Therefore, a dialysis membrane is used for separation, so that the released DHM molecules diffuse through the membrane and the nanodroplets remain inside the membrane.) The sealed dialysis tubing is submerged in a solution of 1.6 mL water and 27 mL 1.1× strength FaSSGF (fasted state simulated gastric fluid) for 30 min. (“1.1× strength FaSSGF” is made by mixing 0.066 g/L of “Biorelevant FaSSIF/FeSSIF/FaSSIF” powder, 2.20 g/L sodium chloride, and water, and adjusting the pH to 1.6 using hydrochloric acid.) An aliquot was taken from the bulk medium outside of the dialysis membrane (this aliquot allows determination of the amount of DHM released from the nanodroplets). At the end of 30 min, the dialysis membrane was dissembled, and the liquid was remixed (i.e., the NE-5 within the membrane was mixed with the water and FaSSGF and released DHM outside of the membrane). The reason for this remixing is to make the mixture homogenous again, so as to simulate the scenario in which the nanodroplets encapsulating DHM and the released DHM molecules flow from a human's stomach after about 30 minutes of residence time into the intestines for another 6 hours of digestion. The DHM is at a concentration of 2 mg/mL in the remixed liquid (which, aside from the DHM, is 1× strength FaSSGF). (The resultant “1× strength FaSSGF” has 0.08 mM taurocholate, 0.02 mM phospholipids, 34 mM sodium, and 59 mM chloride.)

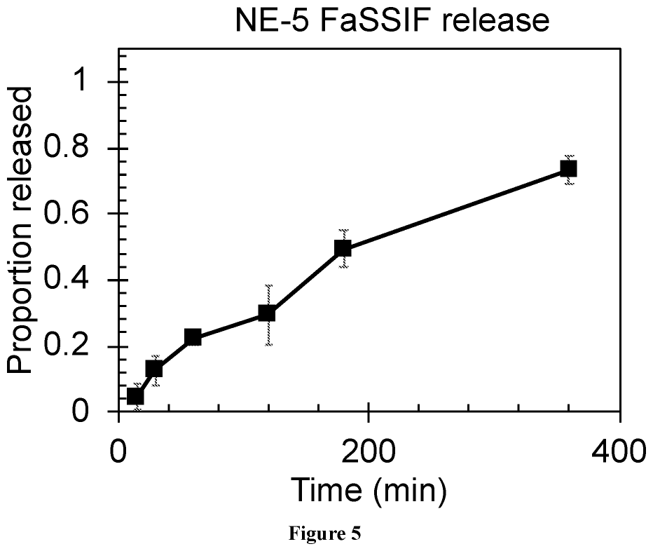

3 mL of the remixed DHM in FaSSGF is sealed into another dialysis membrane, which is transferred to 27 mL of 1.1× strength FaSSIF (fasted state simulated intestinal fluid). (“1.1× strength FaSSIF” can be made by mixing 2.46 g/L of “Biorelevant FaSSIF/FeSSIF/FaSSIF” powder, 0.46 g/L of sodium hydroxide, 4.35 g/L of sodium phosphate monobasic monohydrate, and 6.81 g/L sodium chloride, and water, and adjusting the pH to 6.5 using sodium hydroxide or hydrochloric acid.) Aliquots were taken from the bulk medium outside of the dialysis membrane during the 6-hour duration of the experiment. After the 6 hours the dialysis membrane was disassembled and the liquid was remixed (i.e., the NE-5 and liquid within the membrane was mixed with the FaSSIF and released DHM outside of the membrane). The DHM is at a concentration of 0.2 mg/mL in the remixed liquid (which, aside from the DHM is 1× strength FaSSIF). (The resultant “1× strength FaSSIF” has 3 mM taurocholate, 0.75 mM phospholipids, 148 mM sodium, 106 mM chloride, and 29 mM phosphate.) An aliquot of the remixed liquid is taken to measure the total concentration of DHM as a reference value (this reference value is the released DHM that has diffused through the dialysis membrane plus the remaining DHM still encapsulated within the nanodroplets). The percentage of release of DHM through the dialysis membrane at a given time is defined as the DHM concentration at that time in the medium outside of the dialysis membrane over this reference value. In a human, the released DHM (outside of the nanodroplets) can then diffuse through the intestinal wall and enter into the blood circulation. The results are plotted in FIG. 5. The results show that about 70% of the DHM in the nanodroplets is gradually released from the nanodroplets over 6 hours in FaSSIF.

### Example 7B

13% Capmul MCM (2,3-Dihydroxypropyl octanoate), 26% Kolliphor EL (polyethoxylated castor oil), 13% Transcutol (2-(2-ethoxyethoxy)ethanol), 4.3% pure dihydromyricetin (DHM) crystals, and 43.7% water are mixed at 40° C. to make formulation NE-5. 1.4 g of NE-5 is placed into 6-8kD dialysis tubing, which is sealed at both ends. The sealed dialysis tubing is submerged in 1.6 mL water and 27 ml 1.1× strength FaSSGF for 30 min. An aliquot was taken from the bulk medium outside of the dialysis membrane. At the end of 30 min, the dialysis membrane was dissembled, and the liquid was remixed (i.e., the NE-5 within the membrane was mixed with the water and FaSSGF and released DHM outside of the membrane). The DHM is at a concentration of 2 mg/mL in the remixed liquid (which, aside from the DHM, is 1× strength FaSSGF).

3 mL of the remixed DHM in FaSSGF is sealed into another dialysis membrane, which is transferred to 27 mL of 1.1× strength FeSSIF (fed state simulated intestinal fluid). (“1.1× strength FeSSIF” can be made by mixing 10.7 g/L of FeSSIF-V2 powder, 3.60 g/L sodium hydroxide, 7.03 g/L maleic acid, 8.06 g/L sodium chloride, and water, and adjusting the pH to 5.8.)

Aliquots were taken from the bulk medium outside of the dialysis membrane during the 6-hour duration of the experiment. After the 6 hours the dialysis membrane was disassembled and the liquid was remixed (i.e., the NE-5 and liquid within the membrane was mixed with the FeSSIF and released DHM outside of the membrane). The DHM is at a concentration of 0.2 mg/mL in the remixed liquid (which, aside from the DHM is 1× strength FeSSIF). (The resultant “1× strength FeSSIF” has 15 mM taurocholate, 3.75 mM phospholipids, 319 mM sodium, 203 mM chloride, and 144 mM organic acid.)

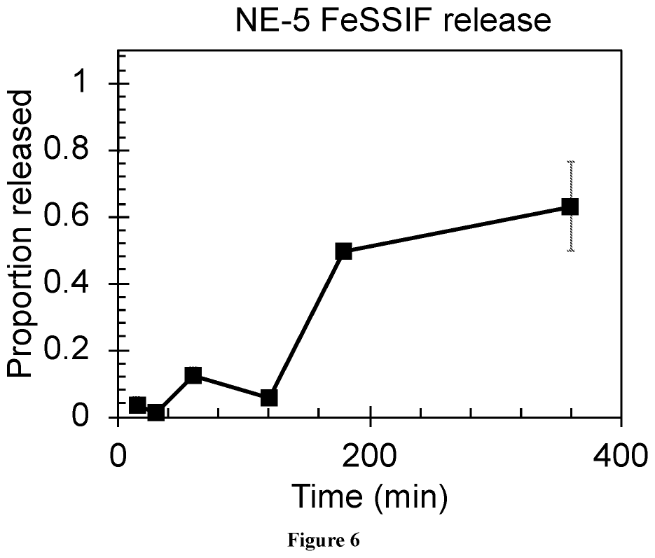

An aliquot of the remixed liquid is taken to measure the total concentration of DHM as a reference value (this reference value is the released DHM that has diffused through the dialysis membrane plus the remaining DHM still encapsulated within the nanodroplets). The percentage of release of DHM through the dialysis membrane at a given time is defined as the DHM concentration at that time in the medium outside of the dialysis membrane over this reference value. In a human, the released DHM (outside of the nanodroplets) can then diffuse through the intestinal wall and enter into the blood circulation. The results are plotted in FIG. 6. The results show that about 60% of the DHM in the nanodroplets is gradually released from the nanodroplets over 6 hours in FeSSIF.

### Example 8

A no-water formulation is prepared with 19.4 wt % Capmul MCM (2,3-dihydroxypropyl octanoate), 38.8 wt % Kolliphor EL (polyethoxylated castor oil), 19.4 wt % transcutol (2-(2-ethoxyethoxy)ethanol), and 22.4 wt % pure dihydromyricetin (DHM) crystals. This formulation is mixed at 40° C. to give a clear and homogenous liquid. Each size 9 softgel capsule without an enteric coating is loaded with 31 +/−1 mg of formulation, which includes 7 mg of DHM.

Preliminary in vitro experiments are done by placing 2 loaded capsules into 0.2 mL of water and 1.8 mL of 1.1× strength FaSSGF (approximate volume of rat stomach). The capsules dissolved within 5 minutes. At the end of 30 minutes, dynamic light scattering results show that the no-water formulation emulsified in water to give nanodroplets of an mean diameter of 500 nm. 1 mL of this nanoemulsion in FaSSGF is transferred to 9 mL of 1.1633 strength FeSSIF buffer and at the end of 18 hours, nanodroplets having a mean diameter of 200 nm are observed by dynamic light scattering.

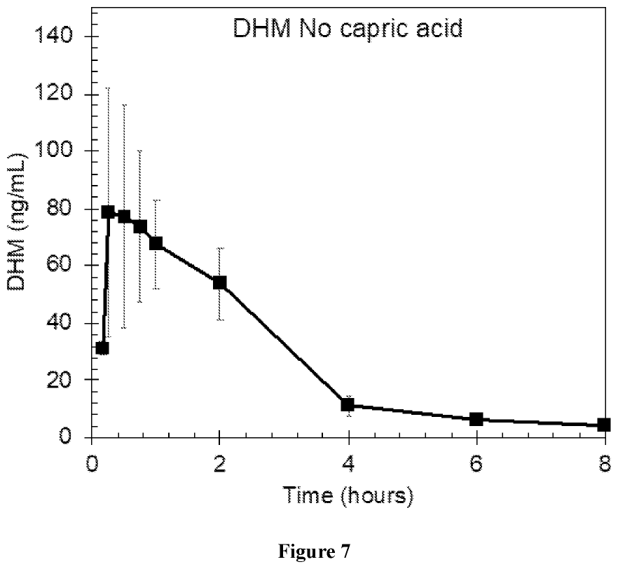

For the in vivo experiment, the total dosing for each rat is 2 capsules, so that each rat is administered 14 mg DHM in total. Each rat weighed 300 +/−50 g. For each sample, blood is drawn by a catheter from the tail vein of a rat. Plasma is prepared from blood and the amount of DHM is determined by LC/MS (liquid chromatography/mass spectrometry). The in vivo results of DHM in plasma are shown in FIG. 7.

### Example 9

A no-water formulation is prepared with 13.9 wt % Capmul MCM (2,3-dihydroxypropyl octanoate), 27.8 wt % Kolliphor EL (polyethoxylated castor oil), 13.9 wt % transcutol (2-(2-ethoxyethoxy)ethanol), 16.0 wt % pure dihydromyricetin (DHM) crystals, and 28.4 wt % capric acid. This formulation is mixed at 40° C. Each size 9 softgel capsule without an enteric coating is loaded with 29 +/−1 mg of formulation, which includes approximately 4.6 mg of DHM and 8.2 mg of capric acid. Preliminary in vitro experiments are done by placing 3 loaded capsules into 0.2 mL of water and 1.8 mL of 1.1× strength FaSSGF (approximate volume of rat stomach). The capsules dissolved within 5 minutes. At the end of 30 minutes, dynamic light scattering results show that no-water formulation emulsified in water to give nanodroplets of a mean diameter of 1500 nm. 1 mL of this nanoemulsion in FaSSGF is transferred to 9 mL of 1.1× strength FeSSIF buffer and at the end of 18 hours, nanodroplets having a mean diameter of 600 nm are observed by dynamic light scattering.

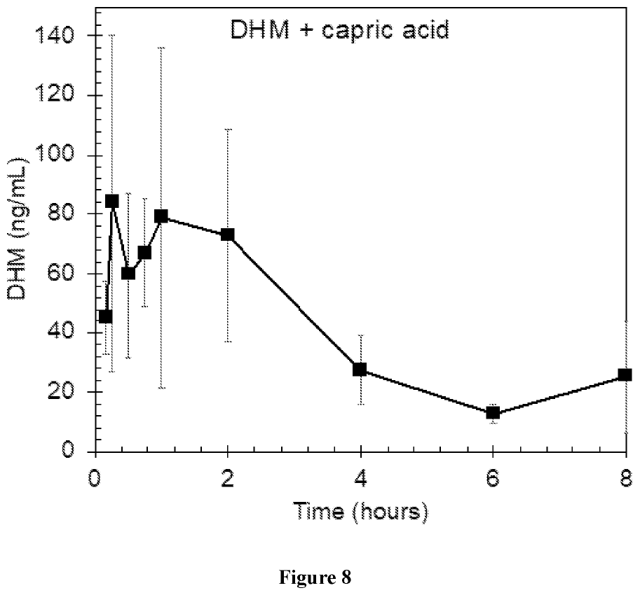

For the in vivo experiment, the total dosing for each rat is 3 capsules, so that each rat is administered 14 mg of DHM and 25 mg of capric acid in total. Each rat weighed 300 +/−50 g. For each sample, blood is drawn by a catheter from the tail vein of a rat. Plasma is prepared from blood and the amount of DHM is determined by LC/MS (liquid chromatography/mass spectrometry). The in vivo results of DHM in plasma are shown in FIG. 8.

Comparing the results of Example 8 (FIG. 7) and Example 9 (FIG. 8), the addition of the permeabilizer-enhancer capric acid results in a higher area under curve (AUC) of DHM in the blood of rats. That is, the DHM concentration in blood decreases more slowly with time for rats administered the formulation with capric acid compared to rats administered the formulation without capric acid.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

### REFERENCES

1. Powell, B. A. R., COMPOSITIONS AND METHODS FOR PREVENTING AND RECOVERY FROM DETRIMIENTAL EFFECTS OF ALCOHOL CONSUMPTION, in Google Patents. 2017, THRIVEPLUS LLC: USA.

2. Shen, Y., et al., Dihydromyricetin As a Novel Anti-Alcohol Intoxication Medication. The

Journal of Neuroscience, 2012. 32(1): p. 390-401.

3. Anton, N., et al., Nano-emulsions and nanocapsules by the PIT method: an investigation on the role of the temperature cycling on the emulsion phase inversion. International Journal of Pharmaceutics, 2007. 344(1-2): p. 44-52.

4. Anton, N. and T. F. Vandamme, The universality of low-energy nano-emulsification. International Journal of Pharmaceutics, 2009. 377(1-2): p. 142-147.

5. Bouchemal, K., et al., Nano-emulsion formulation using spontaneous emulsification: solvent, oil and surfactant optimisation. International journal of pharmaceutics, 2004. 280(1-2): p. 241-251.

6. Fernandez, P., et al., Nano-emulsion formation by emulsion phase inversion. Colloids and Surfaces A: Physicochemical and Engineering Aspects, 2004. 251(1-3): p. 53-58.

7. Izquierdo, P., et al., Formation and stability of nano-emulsions prepared using the phase inversion temperature method. Langmuir, 2002. 18(1): p. 26-30.

8. Rang, M.-J. and C. A. Miller, Spontaneous emulsification of oils containing hydrocarbon, nonionic surfactant, and oleyl alcohol. Journal of colloid and interface science, 1999. 209(1): p. 179-192.

9. Roger, K., B. Cabane, and U. Olsson, Emulsification through rapid curvature changes: The PIT and PIC methods revisited.

10. Roger, K., B. Cabane, and U. Olsson, Formation of 10-100 nm size-controlled emulsions through a sub-PIT cycle. Langmuir, 2009. 26(6): p. 3860-3867.

11. Roger, K., B. Cabane, and U. Olsson, Emulsification through surfactant hydration: the PIC process revisited. Langmuir, 2010. 27(2): p. 604-611.

12. Solans, C., et al., Nano-emulsions. Current opinion in colloid & interface science, 2005. 10(3-4): p. 102-110.

13. Taisne, L. and B. Cabane, Emulsification and ripening following a temperature quench. Langmuir, 1998. 14(17): p. 4744-4752.

14. Gupta, A., et al., Nanoemulsions: formation, properties and applications. Soft Matter, 2016. 12(11): p. 2826-2841.

15. Weder, H. G. and M. Mutsch, Process for the production of an emulsion of oil particles in an aqueous phase. 1992, Hans Georg Weder.

16. Simonnet, J.-T., O. Sonneville, and S. Legret, Nanoemulsion based on sugar fatty esters or on sugar fatty ethers and its uses in the cosmetics, dermatological and/or ophthalmological fields in Google Patents. 2004, L'Oreal SA.

17. Simonnet, J.-T., O. Sonneville, and S. Legret, Nanoemulsion based on oxyethylenated or non-oxyethylenated sorbitan fatty esters, and its uses in the cosmetics, dermatological and/or ophthalmological fields in Google Patents. 2002, L'Oreal SA.

18. Simonnet, J.-T., O. Sonneville, and S. Legret, Nanoemulsion based on phosphoric acid fatty acid esters and its uses in the cosmetics, dermatological, pharmaceutical, and/or ophthalmological fields in Google Patents. 2001, L'Oreal SA.

19. Simonnet, J.-T., O. Sonneville, and S. Legret, Nanoemulsion based on ethoxylated fatty ethers or on ethoxylated fatty esters and its uses in the cosmetics, dermatological and/or ophthalmological fields 2002.

20. L'Alloret, F., O. Aubrun-Sonneville, and J.-T. Simonnet, Nanoemulsion containing nonionic polymers, and its uses 2006, L'Oreal SA.

21. Quemin, E., Translucent nanoemulsion, production method, and uses thereof in the cosmetic, dermatological and/or ophthalmological fields 2005, L'Oreal SA.

22. Simonnet, J.-T., O. Sonneville, and S. Legret, Nanoemulsion based on alkyl ether citrates and its uses in the cosmetics, dermatological, pharmacological and/or ophthalmological fields 2002, L'Oreal SA.

23. Wooster, T. J., H. F. Andrews, and P. Sanguansri, Nanoemulsions. 2009, Commonwealth

Scientific And Industrial Research Organisation.

24. Nicolosi, R. J., et al., Compositions and methods for the preparation of nanoemulsions. 2011, University of Massachusetts.

25. Ochomogo, M., et al., Microemulsion Concentrates and Nanoemulsion Flavorant

Compositions For Food Applications 2011, Clorox Co.

26. Ochomogo, M. G., et al., Adjuvant phase inversion concentrated nanoemulsion compositions. 2011, Clorox Co.

27. Brito, L., et al., Oil/surfactant mixtures for self-emulsification. 2015, Glaxosmithkline Biologicals SA.

28. Zhou, K., G. G. Sacripante, and E. G. Zwartz, Solvent-free toner making process using phase inversion 2011, Xerox Corp.

29. Davies, D. L., et al., Recent advances in the discovery and preclinical testing of novel compounds for the prevention and/or treatment of alcohol use disorders. Alcoholism: Clinical and Experimental Research, 2013. 37(1): p. 8-15.

30. Liang, J., et al., Dihydromyricetin prevents fetal alcohol exposure-induced behavioral and physiological deficits: the roles of GABAA receptors in adolescence. Neurochemical research, 2014. 39(6): p. 1147-1161.

31. Shen, Y., et al., Dihydromyricetin as a novel anti-alcohol intoxication medication. Journal of Neuroscience, 2012. 32(1): p. 390-401.

32. Ji, Y., J. Li, and P. Yang, Effects of fruits of Hovenia dulcis Thunb on acute alcohol toxicity in mice. Zhong yao cai=Zhongyaocai=Journal of Chinese medicinal materials, 2001. 24(2): p. 126-128.

33. Fang, H.-L., et al., Treatment of chronic liver injuries in mice by oral administration of ethanolic extract of the fruit of Hovenia dulcis. The American journal of Chinese medicine, 2007. 35(04): p. 693-703.

34. Hase, K., et al., Hepatoprotective Effect of Hovenia dulcis THUNB. on Experimental Liver Injuries Induced by Carbon Tetrachloride or D-Galactosamine: Lipopolysaccharide. Biological and pharmaceutical Bulletin, 1997. 20(4): p. 381-385.

35. Ji, Y., et al., Effects of Hovenia dulcis Thunb on blood sugar and hepatic glycogen in diabetic mice. Zhong yao cai=Zhongyaocai=Journal of Chinese medicinal materials, 2002. 25(3): p. 190-191.

36. Okuma, Y., et al., Effect of extracts from Hovenia dulcis Thunb. alcohol concentration in rats and men administered alcohol. Journal of Japanese Society of Nutrition and Food Science (Japan), 1995.

37. WANG, X.-y. and Z.-t. JIANG, RESEARCH PROGRESS IN NATURAL ANTIOXIDANT DIHYDROMYRICETIN [J]. Food Research and Development, 2007. 2: p. 056.

38. Zhang, X., et al., Scavenging effect of dihydromyricetin on the free radicals by ESR. Modern Food Science and Technology, 2010. 26(10): p. 1040-1042, 1070.

39. Liu, B., et al., Characterization and antioxidant activity of dihydromyricetin-lecithin complex. European Food Research and Technology, 2009. 230(2): p. 325-331.

40. Aungst, B. J., Absorption enhancers: applications and advances. The AAPS journal, 2012. 14(1): p. 10-18.

41. Thanou, M., J. Verhoef, and H. Junginger, Oral drug absorption enhancement by chitosan and its derivatives. Advanced drug delivery reviews, 2001. 52(2): p. 117-126.

42. Whitehead, K., N. Karr, and S. Mitragotri, Safe and effective permeation enhancers for oral drug delivery. Pharmaceutical research, 2008. 25(8): p. 1782-1788.

43. Whitehead, K. and S. Mitragotri, Mechanistic analysis of chemical permeation enhancers for oral drug delivery. Pharmaceutical research, 2008. 25(6): p. 1412-1419.

44. Vertommen, J. and M. S. Martina, Enteric coating for soft capsule 2013, Capsugel.

45. Zhang, Y., et al., Design and Solidification of Fast-Releasing Clofazimine Nanoparticles for Treatment of Cryptosporidiosis. Molecular pharmaceutics, 2017. 14(10): p. 3480-3488.

46. Onoue, S., et al., Self-micellizing solid dispersion of cyclosporine A with improved dissolution and oral bioavailability. Eur J Pharm Sci, 2014. 62: p. 16-22.

A. Ganachaud, F. and J. L. Katz, Nanoparticles and nanocapsules created using the ouzo effect: spontaneous emulsification as an alternative to ultrasonic and high shear devices. ChemPhysChem, 2005. 6(2): p. 209-216.

B. Kelmann, R. G., et al., Carbamazepine parenteral nanoemulsions prepared by spontaneous emulsification process. International journal of pharmaceutics, 2007. 342(1-2): p. 231-239.

C. Murakami, H., et al., Preparation of poly (DL-lactide-co-glycolide) nanoparticles by modified spontaneous emulsification solvent diffusion method. International journal of pharmaceutics, 1999. 187(2): p. 143-152.

D. Niwa, T., et al., Preparations of biodegradable nanospheres of water-soluble and insoluble drugs with D, L-lactide/glycolide copolymer by a novel spontaneous emulsification solvent diffusion method, and the drug release behavior. Journal of Controlled Release, 1993. 25(1-2): p. 89-98.

E. Ruschak, K. J. and C. A. Miller, Spontaneous emulsification in ternary systems with mass transfer. Industrial & Engineering Chemistry Fundamentals, 1972. 11(4): p. 534-540.

F. Saberi, A. H., Y. Fang, and D. J. McClements, Fabrication of vitamin E-enriched nanoemulsions: factors affecting particle size using spontaneous emulsification. Journal of colloid and interface science, 2013. 391: p. 95-102.

G. Roger, K., et al., Superswollen microemulsions stabilized by shear and trapped by a temperature quench. Langmuir, 2011. 27(17): p. 10447-10454.

1. A dihydromyricetin (DHM) pre-emulsion composition, comprising:
dihydromyricetin (DHM); and
an emulsifier.

2. The DHM pre-emulsion composition of claim 1, wherein the emulsifier comprises a nonionic surfactant.

3. The DHM pre-emulsion composition of claim 1, wherein the emulsifier has a hydrophilic-lipophilic balance (HLB) greater than 7.

4. The DHM pre-emulsion composition of claim 1, wherein the emulsifier comprises a compound selected from the group consisting of a polyethoxylated fatty acid and polyethoxylated castor oil.

5. The DHM pre-emulsion composition of claim 1, wherein the emulsifier comprises a compound selected from the group consisting of a polymeric emulsifier, a polymer of ethylene oxide, a polyethylene glycol sorbitan fatty acid ester (polysorbate), polyoxyethylene sorbitan monolaurate (Tween 20), polyoxyethylene sorbitan monopalmitate (Tween 40), polyoxyethylene sorbitan monostearate (Tween 60), polyoxyethylene sorbitan tristearate (Tween 65), polyoxyethylene sorbitan mono-oleate (Tween 80), and combinations.

6. The DHM pre-emulsion composition of claim 1, wherein the emulsifier comprises a compound selected from the group consisting of a polyethoxyethylene sorbitan monoester, a sugar surfactant, a sugar ester, a sugar fatty acid ester, sucrose monopalmitate, sucrose monolaurate, sucrose monostearate, sucrose distearate, sucrose monopalmitate, sucrose dipalmitate, sucrose monolaurate, saccharose monolaurate, a polyethylene glycol alkyl ether, a polyethylene glycol alkyl phenol, a polyglycerol ester, a polyethoxylated fatty acid diester, a polyethylene glycol fatty acid monoester, a polyethylene glycol fatty acid diester, a polyethylene glycol glycerol fatty acid ester, a fatty acid ester, a fatty alcohol ester, a carboxylic acid ester, a glycerol ester, a mixed ester of a fatty acid, a fatty alcohol, a carboxylic acid, and/or glycerol, and combinations, a citric acid ester of a monoglyceride, an alcohol oil transester, a polyvinyl alcohol, an alkyl cellulose, an alkyl guar, a hydroxyalkyl guar, a C₁-C₂ alkyl cellulose, a C₁-C₃ alkyl guar, a C₁-C₃ hydroxyalkyl guar, a polyvinylpyrrolidone, a polyvinylpyrrolidone copolymer, polyvinylcaprolactam, a polyvinylcaprolactam copolymer, a polyvinyl methyl ether, a polyvinyl methyl ether copolymer, an anionic surfactant, an anionic amphiphilic lipid, a polyacrylic acid, a polyacrylic acid copolymer, polyacrylic acid crosslinked with alkyl sucrose, polyacrylic acid crosslinked with allyl pentaerythritol, poly(acrylic acid-co-alkylacrylate), poly(acrylic acid-co-alkylacrylate) crosslinked with alkyl sucrose, poly(acrylic acid-co-alkylacrylate) crosslinked with allyl pentaerythritol, a polyacrylic acid salt (e.g., sodium polyacrylate), a polyacrylic acid copolymer salt, an alkyl ether citrate, an alkenyl succinate, an alkoxylated alkenyl succinate, an alkoxylated glucose alkenyl succinate, an alkoxylated methylglucose alkenyl succinate, a phosphoric acid fatty ester, alkaline salts of dicetyl and dimyristyl phosphate, alkaline salts of cholesterol sulfate, alkaline salts of cholesterol phosphate, lipoamino acids and their salts, mono- and disodium acylglutamates, disodium salt of N-stearoyl-L-glutamic acid, sodium salts of phosphatidic acid, phospholipids, an alkylsulfonic compound, an alkylsulfonic derivative, a cationic surfactant, a cationic amphiphilic lipid, a quaternary ammonium salt, a fatty amine, a primary fatty amine, a secondary fatty amine, a tertiary fatty amine, a fatty amine salt, a primary fatty amine salt, a secondary fatty amine salt, a tertiary fatty amine salt, and combinations. The DHM pre-emulsion composition of claim 1, further comprising a co-surfactant.

8. The DHM pre-emulsion composition of claim 7, wherein the co-surfactant is selected from the group consisting of 2(2-ethoxyethoxy)ethanol, a sorbitan fatty acid ester, sorbitan monolaurate, sorbitan monopalmitate, sorbitan tristearate, sorbitan monostearate, sorbitan monooleate, sorbitan trioleate, a phospholipid, egg lecithin, soy lecithin, epikuron, topcithin, leciprime, lecisoy, emulfluid, emulpur, metarin, emultop, lecigran, lecimulthin, hydroxylated lecithin, lysophosphatidylcholine, cardiolipin, sphingomyelin, phosphatidylcholine, phosphatidyl ethanolamine, phosphatidic acid, phosphatidyl glycerol, phosphatidyl serine, an ionic surfactant, sodium stearoyl lactylate, calcium stearoyl lactylate, and combinations.

9. The DHM pre-emulsion composition of claim 1, further comprising an oil.

10. The DHM pre-emulsion composition of claim 9, wherein the oil has a molecular weight of less than 400 Da.

11. The DHM pre-emulsion composition of claim 9, wherein the DHM is dispersed or dissolved in the oil.

12. The DHM pre-emulsion composition of claim 1, further comprising a permeabilizer.

13. The DHM pre-emulsion composition of claim 12, wherein the permeabilizer comprises capric acid, a caprate salt, and/or sodium caprate.

14. The DHM pre-emulsion composition of claim 12, wherein the permeabilizer comprises a permeabilizer selected from the group consisting of a fatty acid, a saturated fatty acid, and/or a fatty acid complexed with a cation, such as a metal cation, a metal divalent cation, a magnesium divalent cation, a calcium divalent cation, a zinc divalent cation, an iron divalent cation, a metal trivalent cation, an iron trivalent cation, a fatty acid salt, a fatty acid metallic soap, and combinations.

15. The DHM pre-emulsion composition of claim 1, further comprising an antioxidant.

16. The DHM pre-emulsion composition of claim 1, further comprising an electrolyte and/or a sugar.

17. The DHM pre-emulsion composition of claim 1, further comprising a coactive selected from the group consisting of glutathione, L-cysteine, N-acetyl cysteine (NAC), Prickly Pear extract, Milk Thistle, ginger root, vitamin B, vitamin C, vitamin E, and combinations.

18. The DHM pre-emulsion composition of claim 1, which disperses to emulsion droplets of a mean diameter of at most 1000 nanometers when contacted with an excess aqueous phase.

19. A dosage form, comprising:
the dihydromyricetin (DHM) pre-emulsion composition of claim 1; and
a capsule,
wherein the DHM pre-emulsion composition is encapsulated in the capsule.

20. The dosage form of claim 19, wherein the capsule is a soft gel capsule.

21. The dosage form of claim 19, wherein the capsule comprises a material selected from the group consisting of animal-derived material, gelatin, collagen, plant-derived material, synthetically-produced material, a polysaccharide, a sulfated polysaccharide, a carrageenan, cellulose, a cellulose derivative, starch, a starch derivative, pullulan, polyvinyl alcohol (PVA), polyvinyl alcohol (PVA) copolymer, polyethylene glycol (PEG), hydroxypropyl methylcellulose (HPMC), hydroxypropyl methyl cellulose acetate succinate (HPMCAS), a material of algal origin, and combinations.

22. The dosage form of claim 19,
wherein the capsule is not solubilized or dissolved by an aqueous solution having a pH of at most 3.5 and
wherein the capsule is solubilized or dissolved by an aqueous solution having a pH of at least 5.5.

23. The dosage form of claim 19,
wherein the capsule comprises an exterior surface and
wherein the exterior surface is coated with an enteric coating.

24. The dosage form of claim 23, wherein the enteric coating is selected from the group consisting of a polymeric coating, a methacrylate copolymer coating, a hydroxypropyl methyl cellulose acetate succinate (HPMCAS) coating, and combinations.

25. The DHM pre-emulsion composition of claim 1, further comprising:
soybean oil; and
capric acid, sodium caprate, or a caprate salt,
wherein the emulsifier comprises polyoxyethylene sorbitan monolaurate.

26. The dihydromyricetin (DHM) pre-emulsion composition of claim 25,
wherein the dihydromyricetin (DHM) is at a concentration of from 3 to 30 mg/mL in the soybean oil,
wherein the polyoxyethylene sorbitan monolaurate is at a concentration of from 0.3 to 3 mg/mL in the soybean oil, and
wherein the capric acid, sodium caprate, or caprate salt is at a concentration of from 0.02 mg/mL to 0.2 mg/mL in the soybean oil.

27. The DHM pre-emulsion composition of claim 1, further comprising:
2,3-dihydroxypropyl octanoate; and
2-(2-ethoxyethoxy)ethanol,
wherein the emulsifier comprises polyethoxylated castor oil.

28. The dihydromyricetin (DHM) pre-emulsion composition of claim 27, further comprising capric acid, sodium caprate, and/or a caprate salt.

29. The dihydromyricetin (DHM) pre-emulsion composition of claim 27, further comprising water.

30. The dihydromyricetin (DHM) pre-emulsion composition of claim 27, comprising
from 10 wt % to 24 wt % dihydromyricetin (DHM);
from 9 wt % to 21 wt % 2,3-dihydroxypropyl octanoate;
from 9 wt % to 21 wt % 2-(2-ethoxyethoxy)ethanol; and
from 0 wt % to 34 wt % capric acid, sodium caprate, and/or a caprate salt,
wherein the balance comprises polyethoxylated castor oil.

31. The dihydromyricetin (DHM) pre-emulsion composition of claim 27, comprising
from 2 wt % to 20 wt % dihydromyricetin (DHM);
from 7 wt % to 20 wt % 2,3-dihydroxypropyl octanoate;
from 3 wt % to 20 wt % 2-(2-ethoxyethoxy)ethanol; and
from 14 wt % to 40 wt % polyethoxylated castor oil,
wherein the balance comprises water.

32. A method for forming the dosage form of claim 19, comprising:
mixing the dihydromyricetin (DHM), the emulsifier, and an oil to dissolve or disperse the DHM and the emulsifier in the oil to form the DHM pre-emulsion composition;
loading the DHM pre-emulsion composition into the capsule; and
sealing the capsule.

33. A method for administering dihydromyricetin (DHM) to a patient, comprising:
orally administering the dosage form of claim 19 to the patient;
allowing the capsule to enter the patient's stomach, where the capsule is not dissolved and is not solubilized by gastric juices in the stomach;
allowing the capsule to pass from the stomach to the patient's intestine, where the capsule is dissolved or solubilized by intestinal fluid in the intestine;
allowing the partially or fully dissolved or solubilized capsule to release the DHM pre-emulsion composition into the intestinal fluid;
allowing the released DHM pre-emulsion composition to form a metastable nanoemulsion comprising oil-in-water droplets in the intestinal fluid; and
allowing the DHM to diffuse from the oil-in-water droplets into a wall of the intestine and into the patient's bloodstream, so that the DHM is administered to the patient.

34. A method for reducing hangover symptoms, comprising
administering the dihydromyricetin (DHM) pre-emulsion composition of claim 1 to a patient suffering from hangover symptoms,
so that the patient's hangover symptoms are reduced.

35. The dihydromyricetin (DHM) formulation of claim 1 for use in preventing an alcohol use disorder, preventing alcoholism, treating an alcohol use disorder, treating alcoholism, or treating an alcohol overdose.

36. The dihydromyricetin (DHM) formulation of claim 1 for use in increasing antioxidant capacity, neuroprotection, preventing Alzheimer's disease, treating Alzheimer's disease, inhibiting inflammation, protecting the kidney, protecting the liver, preventing or treating cancer, ameliorating a metabolic disorder, preventing diabetes, treating diabetes, or treating a bacterial infection.
